SEQUENCE LISTING (289 sequences)

SEQ ID NO: 1 - protein, 14 aa, Artificial Sequence
  Peptide (1)..(14): synthetic construct
KLAKLAKKLAKLAK

SEQ ID NO: 2 - protein, 14 aa, Artificial Sequence
  Peptide (1)..(14): synthetic construct
KLAKKLAKLAKKLA

SEQ ID NO: 3 - protein, 14 aa, Artificial Sequence
  Peptide (1)..(14): synthetic construct
KAAKKAAKAAKKAA

SEQ ID NO: 4 - protein, 21 aa, Artificial Sequence
  Peptide (1)..(21): synthetic construct
KLGKKLGKLGKKLGKLGKKLG

SEQ ID NO: 5 - protein, 5 aa, Artificial Sequence
  Peptide (1)..(5): synthetic construct
CARAC

SEQ ID NO: 6 - protein, 8 aa, Artificial Sequence
  Peptide (1)..(8): synthetic construct
CPQPRPLC

SEQ ID NO: 7 - protein, 7 aa, Artificial Sequence
  Peptide (1)..(7): synthetic construct
CSVWFGC

SEQ ID NO: 8 - protein, 7 aa, Artificial Sequence
  Peptide (1)..(7): synthetic construct
CMVYFGC

SEQ ID NO: 9 - protein, 9 aa, Artificial Sequence
  Peptide (1)..(9): synthetic construct
CDCRGDCFC

SEQ ID NO: 10 - protein, 6 aa, Artificial Sequence
  Peptide (1)..(6): synthetic construct
GRGESP

SEQ ID NO: 11 - protein, 8 aa, Artificial Sequence
  Peptide (1)..(8): synthetic construct
CNIRRQGC

SEQ ID NO: 12 - DNA, 24 bp, Artificial Sequence
  misc_feature (1)..(24): oligonucleotide
ccctcatagttagcgtaacgatct

SEQ ID NO: 13 - protein, 9 aa, Artificial Sequence
  Peptide (1)..(9): synthetic construct
CGQEISGLC

SEQ ID NO: 14 - protein, 6 aa, Artificial Sequence
  Peptide (1)..(6): synthetic construct
EVISGL

SEQ ID NO: 15 - protein, 6 aa, Artificial Sequence
  Peptide (1)..(6): synthetic construct
LISGVL

SEQ ID NO: 16 - protein, 6 aa, Artificial Sequence
  Peptide (1)..(6): synthetic construct
ELLSGL

SEQ ID NO: 17 - protein, 7 aa, Artificial Sequence
  Peptide (1)..(7): synthetic construct
GRLSGSL

SEQ ID NO: 18 - protein, 9 aa, Artificial Sequence
  Peptide (1)..(9): synthetic construct
CLRSGGLTC

SEQ ID NO: 19 - protein, 6 aa, Artificial Sequence
  Peptide (1)..(6): synthetic construct
LGGLSA

SEQ ID NO: 20 - protein, 9 aa, Artificial Sequence
  Peptide (1)..(9): synthetic construct
CWGGLSGLC

SEQ ID NO: 21 - protein, 6 aa, Artificial Sequence
  Peptide (1)..(6): synthetic construct
CGLSLK

SEQ ID NO: 22 - protein, 6 aa, Artificial Sequence
  Peptide (1)..(6): synthetic construct
GLSARH

SEQ ID NO: 23 - protein, 9 aa, Artificial Sequence
  Peptide (1)..(9): synthetic construct
CEFGLSEVC

SEQ ID NO: 24 - protein, 6 aa, Artificial Sequence
  Peptide (1)..(6): synthetic construct
SKHALE

SEQ ID NO: 25 - protein, 6 aa, Artificial Sequence
  Peptide (1)..(6): synthetic construct
VHALES

SEQ ID NO: 26 - protein, 6 aa, Artificial Sequence
  Peptide (1)..(6): synthetic construct
VFALEG

SEQ ID NO: 27 - protein, 9 aa, Artificial Sequence
  Peptide (1)..(9): synthetic construct
CRIRMSAGC

SEQ ID NO: 28 - protein, 6 aa, Artificial Sequence
  Peptide (1)..(6): synthetic construct
AMSAGV

SEQ ID NO: 29 - protein, 9 aa, Artificial Sequence
  Peptide (1)..(9): synthetic construct
CMIAGLGRC

SEQ ID NO: 30 - protein, 9 aa, Artificial Sequence
  Peptide (1)..(9): synthetic construct
CRVIVGPRC

SEQ ID NO: 31 - protein, 9 aa, Artificial Sequence
  Peptide (1)..(9): synthetic construct
CRVFAGKRC

SEQ ID NO: 32 - protein, 6 aa, Artificial Sequence
  Peptide (1)..(6): synthetic construct
DRVTLG

SEQ ID NO: 33 - protein, 9 aa, Artificial Sequence
  Peptide (1)..(9): synthetic construct
CRVTRGHGC

SEQ ID NO: 34 - protein, 9 aa, Artificial Sequence
  Peptide (1)..(9): synthetic construct
CIRVEAGSC

SEQ ID NO: 35 - protein, 4 aa, Artificial Sequence
  Peptide (1)..(4): synthetic construct
ISGL

SEQ ID NO: 36 - protein, 4 aa, Artificial Sequence
  Peptide (1)..(4): synthetic construct
GGLS

SEQ ID NO: 37 - protein, 4 aa, Artificial Sequence
  Peptide (1)..(4): synthetic construct
HALE

SEQ ID NO: 38 - protein, 4 aa, Artificial Sequence
  Peptide (1)..(4): synthetic construct
MSAG

SEQ ID NO: 39 - protein, 5 aa, Artificial Sequence
  MISC_FEATURE (4)..(4): Unidentified aminio acid
RVTXG

SEQ ID NO: 40 - protein, 8 aa, Artificial Sequence
  Peptide (1)..(8): synthetic construct
CEGESASC

SEQ ID NO: 41 - protein, 8 aa, Artificial Sequence
  Peptide (1)..(8): synthetic construct
CVPMRLQC

SEQ ID NO: 42 - protein, 8 aa, Artificial Sequence
  Peptide (1)..(8): synthetic construct
CLGKGSVC

SEQ ID NO: 43 - protein, 8 aa, Artificial Sequence
  Peptide (1)..(8): synthetic construct
CLSPIGEC

SEQ ID NO: 44 - protein, 8 aa, Artificial Sequence
  Peptide (1)..(8): synthetic construct
CNLSVPAC

SEQ ID NO: 45 - protein, 8 aa, Artificial Sequence
  Peptide (1)..(8): synthetic construct
CIIGSYVC

SEQ ID NO: 46 - protein, 8 aa, Artificial Sequence
  Peptide (1)..(8): synthetic construct
CADVLRPC

SEQ ID NO: 47 - protein, 8 aa, Artificial Sequence
  Peptide (1)..(8): synthetic construct
CWRSVEVC

SEQ ID NO: 48 - protein, 8 aa, Artificial Sequence
  Peptide (1)..(8): synthetic construct
CSIRRESC

SEQ ID NO: 49 - protein, 8 aa, Artificial Sequence
  Peptide (1)..(8): synthetic construct
CAVVFSQC

SEQ ID NO: 50 - protein, 8 aa, Artificial Sequence
  Peptide (1)..(8): synthetic construct
CLANLQTC

SEQ ID NO: 51 - protein, 8 aa, Artificial Sequence
  Peptide (1)..(8): synthetic construct
CNIRRQGC

SEQ ID NO: 52 - protein, 8 aa, Artificial Sequence
  Peptide (1)..(8): synthetic construct
CIRREKRC

SEQ ID NO: 53 - protein, 8 aa, Artificial Sequence
  Peptide (1)..(8): synthetic construct
CAGKSSNC

SEQ ID NO: 54 - protein, 8 aa, Artificial Sequence
  Peptide (1)..(8): synthetic construct
CRECGERC

SEQ ID NO: 55 - protein, 8 aa, Artificial Sequence
  Peptide (1)..(8): synthetic construct
CMARQARC

SEQ ID NO: 56 - protein, 8 aa, Artificial Sequence
  Peptide (1)..(8): synthetic construct
CLPISSSC

SEQ ID NO: 57 - protein, 8 aa, Artificial Sequence
  Peptide (1)..(8): synthetic construct
CGRAKVRC

SEQ ID NO: 58 - protein, 8 aa, Artificial Sequence
  Peptide (1)..(8): synthetic construct
CASGSENC

SEQ ID NO: 59 - protein, 8 aa, Artificial Sequence
  Peptide (1)..(8): synthetic construct
CMRGKGLC

SEQ ID NO: 60 - protein, 8 aa, Artificial Sequence
  Peptide (1)..(8): synthetic construct
CAGGGAYC

SEQ ID NO: 61 - protein, 8 aa, Artificial Sequence
  Peptide (1)..(8): synthetic construct
CAAAPIRC

SEQ ID NO: 62 - protein, 8 aa, Artificial Sequence
  Peptide (1)..(8): synthetic construct
CGRDSKQC

SEQ ID NO: 63 - protein, 9 aa, Artificial Sequence
  Peptide (1)..(9): synthetic construct
CLGRLTVLC

SEQ ID NO: 64 - protein, 9 aa, Artificial Sequence
  Peptide (1)..(9): synthetic construct
CTAWFIESC

SEQ ID NO: 65 - protein, 9 aa, Artificial Sequence
  Peptide (1)..(9): synthetic construct
CSYGRASLC

SEQ ID NO: 66 - protein, 9 aa, Artificial Sequence
  Peptide (1)..(9): synthetic construct
CDAGPWTAC

SEQ ID NO: 67 - protein, 9 aa, Artificial Sequence
  Peptide (1)..(9): synthetic construct
CVGVGRSRC

SEQ ID NO: 68 - protein, 9 aa, Artificial Sequence
  Peptide (1)..(9): synthetic construct
CTNPWSPVC

SEQ ID NO: 69 - protein, 9 aa, Artificial Sequence
  Peptide (1)..(9): synthetic construct
CGGSYDEVC

SEQ ID NO: 70 - protein, 9 aa, Artificial Sequence
  Peptide (1)..(9): synthetic construct
CAPMEWSVC

SEQ ID NO: 71 - protein, 9 aa, Artificial Sequence
  Peptide (1)..(9): synthetic construct
CTRVHGLAC

SEQ ID NO: 72 - protein, 9 aa, Artificial Sequence
  Peptide (1)..(9): synthetic construct
CESLSHVDC

SEQ ID NO: 73 - protein, 9 aa, Artificial Sequence
  Peptide (1)..(9): synthetic construct
CLWTQSSGC

SEQ ID NO: 74 - protein, 9 aa, Artificial Sequence
  Peptide (1)..(9): synthetic construct
CERSIGFAC

SEQ ID NO: 75 - protein, 9 aa, Artificial Sequence
  Peptide (1)..(9): synthetic construct
CSVPVSSSC

SEQ ID NO: 76 - protein, 9 aa, Artificial Sequence
  Peptide (1)..(9): synthetic construct
CYPGYDSYC

SEQ ID NO: 77 - protein, 9 aa, Artificial Sequence
  Peptide (1)..(9): synthetic construct
CPWYWFGTC

SEQ ID NO: 78 - protein, 9 aa, Artificial Sequence
  Peptide (1)..(9): synthetic construct
CECRGDCYC

SEQ ID NO: 79 - protein, 9 aa, Artificial Sequence
  Peptide (1)..(9): synthetic construct
CVKKGGFWC

SEQ ID NO: 80 - protein, 9 aa, Artificial Sequence
  Peptide (1)..(9): synthetic construct
CSMTKLGAC

SEQ ID NO: 81 - protein, 9 aa, Artificial Sequence
  Peptide (1)..(9): synthetic construct
CGVLKPYLC

SEQ ID NO: 82 - protein, 9 aa, Artificial Sequence
  Peptide (1)..(9): synthetic construct
CPWYWFGTC

SEQ ID NO: 83 - protein, 9 aa, Artificial Sequence
  Peptide (1)..(9): synthetic construct
CWVADGYRC

SEQ ID NO: 84 - protein, 9 aa, Artificial Sequence
  Peptide (1)..(9): synthetic construct
CECRGDCYC

SEQ ID NO: 85 - protein, 9 aa, Artificial Sequence
  Peptide (1)..(9): synthetic construct
CSHAVMPWC

SEQ ID NO: 86 - protein, 9 aa, Artificial Sequence
  Peptide (1)..(9): synthetic construct
CSERIARVC

SEQ ID NO: 87 - protein, 9 aa, Artificial Sequence
  Peptide (1)..(9): synthetic construct
CPWYWLGWC

SEQ ID NO: 88 - protein, 9 aa, Artificial Sequence
  Peptide (1)..(9): synthetic construct
CGRKNEWAC

SEQ ID NO: 89 - protein, 9 aa, Artificial Sequence
  Peptide (1)..(9): synthetic construct
CARDRIIAC

SEQ ID NO: 90 - protein, 9 aa, Artificial Sequence
  Peptide (1)..(9): synthetic construct
CGQMNREVC

SEQ ID NO: 91 - protein, 9 aa, Artificial Sequence
  Peptide (1)..(9): synthetic construct
CDAYPLFFC

SEQ ID NO: 92 - protein, 9 aa, Artificial Sequence
  Peptide (1)..(9): synthetic construct
CWKGFGWWC

SEQ ID NO: 93 - protein, 9 aa, Artificial Sequence
  Peptide (1)..(9): synthetic construct
CLGSGSGSC

SEQ ID NO: 94 - protein, 9 aa, Artificial Sequence
  Peptide (1)..(9): synthetic construct
CWWPWGWGC

SEQ ID NO: 95 - protein, 9 aa, Artificial Sequence
  Peptide (1)..(9): synthetic construct
CSWWTFGFC

SEQ ID NO: 96 - protein, 9 aa, Artificial Sequence
  Peptide (1)..(9): synthetic construct
CNSRAGSVC

SEQ ID NO: 97 - protein, 9 aa, Artificial Sequence
  Peptide (1)..(9): synthetic construct
CLRLSMSAC

SEQ ID NO: 98 - protein, 9 aa, Artificial Sequence
  Peptide (1)..(9): synthetic construct
CNSRAGSVC

SEQ ID NO: 99 - protein, 9 aa, Artificial Sequence
  Peptide (1)..(9): synthetic construct
CMSGNTERC

SEQ ID NO: 100 - protein, 9 aa, Artificial Sequence
  Peptide (1)..(9): synthetic construct
CGHLGSVYC

SEQ ID NO: 101 - protein, 9 aa, Artificial Sequence
  Peptide (1)..(9): synthetic construct
CVLADPTGC

SEQ ID NO: 102 - protein, 9 aa, Artificial Sequence
  Peptide (1)..(9): synthetic construct
CECRGDCYC

SEQ ID NO: 103 - protein, 9 aa, Artificial Sequence
  Peptide (1)..(9): synthetic construct
CWWGWWGTC

SEQ ID NO: 104 - protein, 9 aa, Artificial Sequence
  Peptide (1)..(9): synthetic construct
CWKGFGWWC

SEQ ID NO: 105 - protein, 9 aa, Artificial Sequence
  Peptide (1)..(9): synthetic construct
CKRSATILC

SEQ ID NO: 106 - protein, 9 aa, Artificial Sequence
  Peptide (1)..(9): synthetic construct
CIEGRRGLC

SEQ ID NO: 107 - protein, 9 aa, Artificial Sequence
  Peptide (1)..(9): synthetic construct
CPWYWLGWC

SEQ ID NO: 108 - protein, 9 aa, Artificial Sequence
  Peptide (1)..(9): synthetic construct
CVRQGEDAC

SEQ ID NO: 109 - protein, 9 aa, Artificial Sequence
  Peptide (1)..(9): synthetic construct
CSLAVPLAC

SEQ ID NO: 110 - protein, 9 aa, Artificial Sequence
  Peptide (1)..(9): synthetic construct
CMMHGLAAC

SEQ ID NO: 111 - protein, 9 aa, Artificial Sequence
  Peptide (1)..(9): synthetic construct
CDWWTTAWC

SEQ ID NO: 112 - protein, 9 aa, Artificial Sequence
  Peptide (1)..(9): synthetic construct
CGWWGLWPC

SEQ ID NO: 113 - protein, 9 aa, Artificial Sequence
  Peptide (1)..(9): synthetic construct
CGWFSWFGC

SEQ ID NO: 114 - protein, 9 aa, Artificial Sequence
  Peptide (1)..(9): synthetic construct
CRVDFSKGC

SEQ ID NO: 115 - protein, 9 aa, Artificial Sequence
  Peptide (1)..(9): synthetic construct
CSSLATVVC

SEQ ID NO: 116 - protein, 9 aa, Artificial Sequence
  Peptide (1)..(9): synthetic construct
CMYRTSLAC

SEQ ID NO: 117 - protein, 9 aa, Artificial Sequence
  Peptide (1)..(9): synthetic construct
CLAAVYQSC

SEQ ID NO: 118 - protein, 9 aa, Artificial Sequence
  Peptide (1)..(9): synthetic construct
CSRRVIGAC

SEQ ID NO: 119 - protein, 9 aa, Artificial Sequence
  Peptide (1)..(9): synthetic construct
CSWWNWFGC

SEQ ID NO: 120 - protein, 9 aa, Artificial Sequence
  Peptide (1)..(9): synthetic construct
CSRRPEVVC

SEQ ID NO: 121 - protein, 8 aa, Artificial Sequence
  Peptide (1)..(8): synthetic construct
CVTGNRGC

SEQ ID NO: 122 - protein, 9 aa, Artificial Sequence
  Peptide (1)..(9): synthetic construct
CVSWWFWGC

SEQ ID NO: 123 - protein, 9 aa, Artificial Sequence
  Peptide (1)..(9): synthetic construct
CGWFSWWGC

SEQ ID NO: 124 - protein, 9 aa, Artificial Sequence
  Peptide (1)..(9): synthetic construct
CSWWRFGYC

SEQ ID NO: 125 - protein, 16 aa, Artificial Sequence
  Peptide (1)..(16): synthetic construct
RDLLHVLAFSKSCHLP

SEQ ID NO: 126 - protein, 16 aa, Artificial Sequence
  Peptide (1)..(16): synthetic construct
RDLLHLLAFSKSCSLP

SEQ ID NO: 127 - protein, 16 aa, Artificial Sequence
  Peptide (1)..(16): synthetic construct
DLLHLLAFSKSCSLLP

SEQ ID NO: 128 - protein, 8 aa, Artificial Sequence
  Peptide (1)..(8): synthetic construct
CVFAILAC

SEQ ID NO: 129 - protein, 8 aa, Artificial Sequence
  Peptide (1)..(8): synthetic construct
CGVQYVNC

SEQ ID NO: 130 - protein, 8 aa, Artificial Sequence
  Peptide (1)..(8): synthetic construct
CSYKANSC

SEQ ID NO: 131 - protein, 8 aa, Artificial Sequence
  Peptide (1)..(8): synthetic construct
CYQSSSGC

SEQ ID NO: 132 - protein, 8 aa, Artificial Sequence
  Peptide (1)..(8): synthetic construct
CRGGGRLC

SEQ ID NO: 133 - protein, 8 aa, Artificial Sequence
  Peptide (1)..(8): synthetic construct
CGSDRWLC

SEQ ID NO: 134 - protein, 8 aa, Artificial Sequence
  Peptide (1)..(8): synthetic construct
CLVYNPAC

SEQ ID NO: 135 - protein, 8 aa, Artificial Sequence
  Peptide (1)..(8): synthetic construct
CIPGTSLC

SEQ ID NO: 136 - protein, 8 aa, Artificial Sequence
  Peptide (1)..(8): synthetic construct
CATEAVGC

SEQ ID NO: 137 - protein, 8 aa, Artificial Sequence
  Peptide (1)..(8): synthetic construct
CWGGNQAC

SEQ ID NO: 138 - protein, 9 aa, Artificial Sequence
  Peptide (1)..(9): synthetic construct
CTSWWFWSC

SEQ ID NO: 139 - protein, 9 aa, Artificial Sequence
  Peptide (1)..(9): synthetic construct
CEWSGIWAC

SEQ ID NO: 140 - protein, 9 aa, Artificial Sequence
  Peptide (1)..(9): synthetic construct
CNPLFWWWC

SEQ ID NO: 141 - protein, 9 aa, Artificial Sequence
  Peptide (1)..(9): synthetic construct
CGGWLFPPC

SEQ ID NO: 142 - protein, 9 aa, Artificial Sequence
  Peptide (1)..(9): synthetic construct
CEWWPEWLC

SEQ ID NO: 143 - protein, 9 aa, Artificial Sequence
  Peptide (1)..(9): synthetic construct
CARYLWSWC

SEQ ID NO: 144 - protein, 9 aa, Artificial Sequence
  Peptide (1)..(9): synthetic construct
CAWWRFGLC

SEQ ID NO: 145 - protein, 9 aa, Artificial Sequence
  Peptide (1)..(9): synthetic construct
CRGEWGMMC

SEQ ID NO: 146 - protein, 9 aa, Artificial Sequence
  Peptide (1)..(9): synthetic construct
CFWPFESWC

SEQ ID NO: 147 - protein, 9 aa, Artificial Sequence
  Peptide (1)..(9): synthetic construct
CSNAWVHAC

SEQ ID NO: 148 - protein, 9 aa, Artificial Sequence
  Peptide (1)..(9): synthetic construct
CSWYWWLGC

SEQ ID NO: 149 - protein, 9 aa, Artificial Sequence
  Peptide (1)..(9): synthetic construct
CGGWLFPPC

SEQ ID NO: 150 - protein, 9 aa, Artificial Sequence
  Peptide (1)..(9): synthetic construct
CIEWGSRDC

SEQ ID NO: 151 - protein, 9 aa, Artificial Sequence
  Peptide (1)..(9): synthetic construct
CVRSSVVAC

SEQ ID NO: 152 - protein, 9 aa, Artificial Sequence
  Peptide (1)..(9): synthetic construct
CEDSSRANC

SEQ ID NO: 153 - protein, 9 aa, Artificial Sequence
  Peptide (1)..(9): synthetic construct
CGGWLFPPC

SEQ ID NO: 154 - protein, 9 aa, Artificial Sequence
  Peptide (1)..(9): synthetic construct
CLLVGQVRC

SEQ ID NO: 155 - protein, 9 aa, Artificial Sequence
  Peptide (1)..(9): synthetic construct
CPRYLFWLC

SEQ ID NO: 156 - protein, 9 aa, Artificial Sequence
  Peptide (1)..(9): synthetic construct
CYRSAGAGC

SEQ ID NO: 157 - protein, 9 aa, Artificial Sequence
  Peptide (1)..(9): synthetic construct
CGGWLFPPC

SEQ ID NO: 158 - protein, 9 aa, Artificial Sequence
  Peptide (1)..(9): synthetic construct
CTRVGPKRC

SEQ ID NO: 159 - protein, 9 aa, Artificial Sequence
  Peptide (1)..(9): synthetic construct
CKSGQIAVC

SEQ ID NO: 160 - protein, 9 aa, Artificial Sequence
  Peptide (1)..(9): synthetic construct
CWWPWGGWC

SEQ ID NO: 161 - protein, 9 aa, Artificial Sequence
  Peptide (1)..(9): synthetic construct
CDWGLWWLC

SEQ ID NO: 162 - protein, 9 aa, Artificial Sequence
  Peptide (1)..(9): synthetic construct
CRGWADRKC

SEQ ID NO: 163 - protein, 9 aa, Artificial Sequence
  Peptide (1)..(9): synthetic construct
CGGWLFPPC

SEQ ID NO: 164 - protein, 9 aa, Artificial Sequence
  Peptide (1)..(9): synthetic construct
CTQVRFSGC

SEQ ID NO: 165 - protein, 9 aa, Artificial Sequence
  Peptide (1)..(9): synthetic construct
CPWWWFGEC

SEQ ID NO: 166 - protein, 9 aa, Artificial Sequence
  Peptide (1)..(9): synthetic construct
CGGWLFPPC

SEQ ID NO: 167 - protein, 7 aa, Artificial Sequence
  Peptide (1)..(7): synthetic construct
CEKRWGC

SEQ ID NO: 168 - protein, 7 aa, Artificial Sequence
  Peptide (1)..(7): synthetic construct
CKQRGVC

SEQ ID NO: 169 - protein, 7 aa, Artificial Sequence
  Peptide (1)..(7): synthetic construct
CQVRLSC

SEQ ID NO: 170 - protein, 7 aa, Artificial Sequence
  Peptide (1)..(7): synthetic construct
CTWDKRC

SEQ ID NO: 171 - protein, 7 aa, Artificial Sequence
  Peptide (1)..(7): synthetic construct
CTLFRNC

SEQ ID NO: 172 - protein, 7 aa, Artificial Sequence
  Peptide (1)..(7): synthetic construct
CRGSAVC

SEQ ID NO: 173 - protein, 7 aa, Artificial Sequence
  Peptide (1)..(7): synthetic construct
CAISVGC

SEQ ID NO: 174 - protein, 7 aa, Artificial Sequence
  Peptide (1)..(7): synthetic construct
CTNPQRC

SEQ ID NO: 175 - protein, 7 aa, Artificial Sequence
  Peptide (1)..(7): synthetic construct
CDSWPLC

SEQ ID NO: 176 - protein, 7 aa, Artificial Sequence
  Peptide (1)..(7): synthetic construct
CENGSRC

SEQ ID NO: 177 - protein, 7 aa, Artificial Sequence
  Peptide (1)..(7): synthetic construct
CGGSSQC

SEQ ID NO: 178 - protein, 7 aa, Artificial Sequence
  Peptide (1)..(7): synthetic construct
CGREGPC

SEQ ID NO: 179 - protein, 7 aa, Artificial Sequence
  Peptide (1)..(7): synthetic construct
CSGRSGC

SEQ ID NO: 180 - protein, 7 aa, Artificial Sequence
  Peptide (1)..(7): synthetic construct
CQQGRYC

SEQ ID NO: 181 - protein, 7 aa, Artificial Sequence
  Peptide (1)..(7): synthetic construct
CVKQMRC

SEQ ID NO: 182 - protein, 7 aa, Artificial Sequence
  Peptide (1)..(7): synthetic construct
CSVWWGC

SEQ ID NO: 183 - protein, 5 aa, Artificial Sequence
  Peptide (1)..(5): synthetic construct
CSGPC

SEQ ID NO: 184 - protein, 7 aa, Artificial Sequence
  Peptide (1)..(7): synthetic construct
CEGHQSC

SEQ ID NO: 185 - protein, 7 aa, Artificial Sequence
  Peptide (1)..(7): synthetic construct
CNVWYGC

SEQ ID NO: 186 - protein, 7 aa, Artificial Sequence
  Peptide (1)..(7): synthetic construct
CRSPMKC

SEQ ID NO: 187 - protein, 7 aa, Artificial Sequence
  Peptide (1)..(7): synthetic construct
CPTMTEC

SEQ ID NO: 188 - protein, 7 aa, Artificial Sequence
  Peptide (1)..(7): synthetic construct
CSVWFGC

SEQ ID NO: 189 - protein, 7 aa, Artificial Sequence
  Peptide (1)..(7): synthetic construct
CSVWYGC

SEQ ID NO: 190 - protein, 7 aa, Artificial Sequence
  Peptide (1)..(7): synthetic construct
CSVWYGC

SEQ ID NO: 191 - protein, 7 aa, Artificial Sequence
  Peptide (1)..(7): synthetic construct
CWILEQC

SEQ ID NO: 192 - protein, 7 aa, Artificial Sequence
  Peptide (1)..(7): synthetic construct
CMATLRC

SEQ ID NO: 193 - protein, 7 aa, Artificial Sequence
  Peptide (1)..(7): synthetic construct
CRKLGGC

SEQ ID NO: 194 - protein, 7 aa, Artificial Sequence
  Peptide (1)..(7): synthetic construct
CRAREMC

SEQ ID NO: 195 - protein, 7 aa, Artificial Sequence
  Peptide (1)..(7): synthetic construct
CQAWQRC

SEQ ID NO: 196 - protein, 7 aa, Artificial Sequence
  Peptide (1)..(7): synthetic construct
CKDRWGC

SEQ ID NO: 197 - protein, 7 aa, Artificial Sequence
  Peptide (1)..(7): synthetic construct
CYSDKKC

SEQ ID NO: 198 - protein, 7 aa, Artificial Sequence
  Peptide (1)..(7): synthetic construct
CGNHQKC

SEQ ID NO: 199 - protein, 7 aa, Artificial Sequence
  Peptide (1)..(7): synthetic construct
CPNDSLC

SEQ ID NO: 200 - protein, 7 aa, Artificial Sequence
  Peptide (1)..(7): synthetic construct
CQGTWIC

SEQ ID NO: 201 - protein, 7 aa, Artificial Sequence
  Peptide (1)..(7): synthetic construct
CXVWXGC

SEQ ID NO: 202 - protein, 9 aa, Artificial Sequence
  Peptide (1)..(9): synthetic construct
CPEVMGSSC

SEQ ID NO: 203 - protein, 9 aa, Artificial Sequence
  Peptide (1)..(9): synthetic construct
CSSVVRLGC

SEQ ID NO: 204 - protein, 9 aa, Artificial Sequence
  Peptide (1)..(9): synthetic construct
CVGAGLHIC

SEQ ID NO: 205 - protein, 9 aa, Artificial Sequence
  Peptide (1)..(9): synthetic construct
CHLEPDWVC

SEQ ID NO: 206 - protein, 9 aa, Artificial Sequence
  Peptide (1)..(9): synthetic construct
CALGRWDRC

SEQ ID NO: 207 - protein, 9 aa, Artificial Sequence
  Peptide (1)..(9): synthetic construct
CFGGVGSWC

SEQ ID NO: 208 - protein, 9 aa, Artificial Sequence
  Peptide (1)..(9): synthetic construct
CGRRDTVDC

SEQ ID NO: 209 - protein, 9 aa, Artificial Sequence
  Peptide (1)..(9): synthetic construct
CLVLGGYGC

SEQ ID NO: 210 - protein, 9 aa, Artificial Sequence
  Peptide (1)..(9): synthetic construct
CWENRGQFC

SEQ ID NO: 211 - protein, 9 aa, Artificial Sequence
  Peptide (1)..(9): synthetic construct
CREQASTGC

SEQ ID NO: 212 - protein, 9 aa, Artificial Sequence
  Peptide (1)..(9): synthetic construct
CVVKLRNRC

SEQ ID NO: 213 - protein, 9 aa, Artificial Sequence
  Peptide (1)..(9): synthetic construct
CVGLRAPLC

SEQ ID NO: 214 - protein, 9 aa, Artificial Sequence
  Peptide (1)..(9): synthetic construct
CQKVARPGC

SEQ ID NO: 215 - protein, 9 aa, Artificial Sequence
  Peptide (1)..(9): synthetic construct
CQKFARPGC

SEQ ID NO: 216 - protein, 9 aa, Artificial Sequence
  Peptide (1)..(9): synthetic construct
CMWGLSYLC

SEQ ID NO: 217 - protein, 9 aa, Artificial Sequence
  Peptide (1)..(9): synthetic construct
CREQRHNLC

SEQ ID NO: 218 - protein, 9 aa, Artificial Sequence
  Peptide (1)..(9): synthetic construct
CLVLSASAC

SEQ ID NO: 219 - protein, 9 aa, Artificial Sequence
  Peptide (1)..(9): synthetic construct
CLLSGLMGC

SEQ ID NO: 220 - protein, 9 aa, Artificial Sequence
  Peptide (1)..(9): synthetic construct
CRGDTKALC

SEQ ID NO: 221 - protein, 9 aa, Artificial Sequence
  Peptide (1)..(9): synthetic construct
CVSQLGRVC

SEQ ID NO: 222 - protein, 9 aa, Artificial Sequence
  Peptide (1)..(9): synthetic construct
CFVFEAMGC

SEQ ID NO: 223 - protein, 9 aa, Artificial Sequence
  Peptide (1)..(9): synthetic construct
CSVIKRGAC

SEQ ID NO: 224 - protein, 9 aa, Artificial Sequence
  Peptide (1)..(9): synthetic construct
CGGWVDHRC

SEQ ID NO: 225 - protein, 9 aa, Artificial Sequence
  Peptide (1)..(9): synthetic construct
CAVVRNQEC

SEQ ID NO: 226 - protein, 9 aa, Artificial Sequence
  Peptide (1)..(9): synthetic construct
CDSPRRPVC

SEQ ID NO: 227 - protein, 9 aa, Artificial Sequence
  Peptide (1)..(9): synthetic construct
CTFSGHRLC

SEQ ID NO: 228 - protein, 9 aa, Artificial Sequence
  Peptide (1)..(9): synthetic construct
CHTWGGRNC

SEQ ID NO: 229 - protein, 9 aa, Artificial Sequence
  Peptide (1)..(9): synthetic construct
CEGAGLVAC

SEQ ID NO: 230 - protein, 9 aa, Artificial Sequence
  Peptide (1)..(9): synthetic construct
CFPRVWSRC

SEQ ID NO: 231 - protein, 9 aa, Artificial Sequence
  Peptide (1)..(9): synthetic construct
CYWLGGALC

SEQ ID NO: 232 - protein, 9 aa, Artificial Sequence
  Peptide (1)..(9): synthetic construct
CDTNQRVVC

SEQ ID NO: 233 - protein, 9 aa, Artificial Sequence
  Peptide (1)..(9): synthetic construct
CMRVTKTHC

SEQ ID NO: 234 - protein, 9 aa, Artificial Sequence
  Peptide (1)..(9): synthetic construct
CDQNWLVHC

SEQ ID NO: 235 - protein, 9 aa, Artificial Sequence
  Peptide (1)..(9): synthetic construct
CTFSGHRLC

SEQ ID NO: 236 - protein, 9 aa, Artificial Sequence
  Peptide (1)..(9): synthetic construct
CALSAYRVC

SEQ ID NO: 237 - protein, 9 aa, Artificial Sequence
  Peptide (1)..(9): synthetic construct
CGGEEGRRC

SEQ ID NO: 238 - protein, 9 aa, Artificial Sequence
  Peptide (1)..(9): synthetic construct
CAEAGGPDC

SEQ ID NO: 239 - protein, 9 aa, Artificial Sequence
  Peptide (1)..(9): synthetic construct
CIVMLGWRC

SEQ ID NO: 240 - protein, 9 aa, Artificial Sequence
  Peptide (1)..(9): synthetic construct
CGHGVTGRC

SEQ ID NO: 241 - protein, 9 aa, Artificial Sequence
  Peptide (1)..(9): synthetic construct
CERGRGAAC

SEQ ID NO: 242 - protein, 9 aa, Artificial Sequence
  Peptide (1)..(9): synthetic construct
CAAGEGWWC

SEQ ID NO: 243 - protein, 9 aa, Artificial Sequence
  Peptide (1)..(9): synthetic construct
CALSAYRVC

SEQ ID NO: 244 - protein, 9 aa, Artificial Sequence
  Peptide (1)..(9): synthetic construct
CLWPWAGEC

SEQ ID NO: 245 - protein, 9 aa, Artificial Sequence
  Peptide (1)..(9): synthetic construct
CTHATWLVC

SEQ ID NO: 246 - protein, 9 aa, Artificial Sequence
  Peptide (1)..(9): synthetic construct
CSGVSTVRC

SEQ ID NO: 247 - protein, 9 aa, Artificial Sequence
  Peptide (1)..(9): synthetic construct
CLVSYMNGC

SEQ ID NO: 248 - protein, 9 aa, Artificial Sequence
  Peptide (1)..(9): synthetic construct
CVRTSSQWC

SEQ ID NO: 249 - protein, 9 aa, Artificial Sequence
  Peptide (1)..(9): synthetic construct
CLGKGLSSC

SEQ ID NO: 250 - protein, 9 aa, Artificial Sequence
  Peptide (1)..(9): synthetic construct
CFTAVEQGC

SEQ ID NO: 251 - protein, 9 aa, Artificial Sequence
  Peptide (1)..(9): synthetic construct
CGGIGPRFC

SEQ ID NO: 252 - protein, 9 aa, Artificial Sequence
  Peptide (1)..(9): synthetic construct
CVATWCEKC

SEQ ID NO: 253 - protein, 9 aa, Artificial Sequence
  Peptide (1)..(9): synthetic construct
CSSELRAAC

SEQ ID NO: 254 - protein, 9 aa, Artificial Sequence
  Peptide (1)..(9): synthetic construct
CKGSLDEIC

SEQ ID NO: 255 - protein, 9 aa, Artificial Sequence
  Peptide (1)..(9): synthetic construct
CSSVVRLGC

SEQ ID NO: 256 - protein, 9 aa, Artificial Sequence
  Peptide (1)..(9): synthetic construct
CLKTEFTAC

SEQ ID NO: 257 - protein, 9 aa, Artificial Sequence
  Peptide (1)..(9): synthetic construct
CPGRLWRAC

SEQ ID NO: 258 - protein, 9 aa, Artificial Sequence
  Peptide (1)..(9): synthetic construct
CSELGGAGC

SEQ ID NO: 259 - protein, 9 aa, Artificial Sequence
  Peptide (1)..(9): synthetic construct
CLGWRAAAC

SEQ ID NO: 260 - protein, 9 aa, Artificial Sequence
  Peptide (1)..(9): synthetic construct
CGAMWGMGC

SEQ ID NO: 261 - protein, 9 aa, Artificial Sequence
  Peptide (1)..(9): synthetic construct
CIGLSGIEC

SEQ ID NO: 262 - protein, 8 aa, Artificial Sequence
  Peptide (1)..(8): synthetic construct
CQKLGWRV

SEQ ID NO: 263 - protein, 9 aa, Artificial Sequence
  Peptide (1)..(9): synthetic construct
CLEWLQQVC

SEQ ID NO: 264 - protein, 9 aa, Artificial Sequence
  Peptide (1)..(9): synthetic construct
CLVLGEKPC

SEQ ID NO: 265 - protein, 9 aa, Artificial Sequence
  Peptide (1)..(9): synthetic construct
CAAGKGLLC

SEQ ID NO: 266 - protein, 9 aa, Artificial Sequence
  Peptide (1)..(9): synthetic construct
CAAGKDLLC

SEQ ID NO: 267 - protein, 8 aa, Artificial Sequence
  Peptide (1)..(8): synthetic construct
CGAQSPRC

SEQ ID NO: 268 - protein, 9 aa, Artificial Sequence
  Peptide (1)..(9): synthetic construct
CLSSVRGWC

SEQ ID NO: 269 - protein, 9 aa, Artificial Sequence
  Peptide (1)..(9): synthetic construct
CSESQLAWC

SEQ ID NO: 270 - protein, 9 aa, Artificial Sequence
  Peptide (1)..(9): synthetic construct
CSRNSVREC

SEQ ID NO: 271 - protein, 9 aa, Artificial Sequence
  Peptide (1)..(9): synthetic construct
CGLVITATC

SEQ ID NO: 272 - protein, 9 aa, Artificial Sequence
  Peptide (1)..(9): synthetic construct
CPGSVRVQC

SEQ ID NO: 273 - protein, 9 aa, Artificial Sequence
  Peptide (1)..(9): synthetic construct
CRGDTKALC

SEQ ID NO: 274 - protein, 9 aa, Artificial Sequence
  Peptide (1)..(9): synthetic construct
CACVRSRNC

SEQ ID NO: 275 - protein, 9 aa, Artificial Sequence
  Peptide (1)..(9): synthetic construct
CRADSEGVC

SEQ ID NO: 276 - protein, 9 aa, Artificial Sequence
  Peptide (1)..(9): synthetic construct
CNVEASVRC

SEQ ID NO: 277 - protein, 9 aa, Artificial Sequence
  Peptide (1)..(9): synthetic construct
CVGNAKLMC

SEQ ID NO: 278 - protein, 9 aa, Artificial Sequence
  Peptide (1)..(9): synthetic construct
CQKLARAGC

SEQ ID NO: 279 - protein, 9 aa, Artificial Sequence
  Peptide (1)..(9): synthetic construct
CGGRAILLC

SEQ ID NO: 280 - protein, 9 aa, Artificial Sequence
  Peptide (1)..(9): synthetic construct
CQLGRAHGC

SEQ ID NO: 281 - protein, 9 aa, Artificial Sequence
  Peptide (1)..(9): synthetic construct
CGLVITATC

SEQ ID NO: 282 - protein, 9 aa, Artificial Sequence
  Peptide (1)..(9): synthetic construct
CVGATYSRC

SEQ ID NO: 283 - protein, 9 aa, Artificial Sequence
  Peptide (1)..(9): synthetic construct
CSAFSVAYC

SEQ ID NO: 284 - protein, 9 aa, Artificial Sequence
  Peptide (1)..(9): synthetic construct
CLAWEVYLC

SEQ ID NO: 285 - protein, 9 aa, Artificial Sequence
  Peptide (1)..(9): synthetic construct
CQWWLGPLC

SEQ ID NO: 286 - protein, 9 aa, Artificial Sequence
  Peptide (1)..(9): synthetic construct
CSLGSFMGC

SEQ ID NO: 287 - protein, 9 aa, Artificial Sequence
  Peptide (1)..(9): synthetic construct
CVLGEISWC

SEQ ID NO: 288 - protein, 9 aa, Artificial Sequence
  Peptide (1)..(9): synthetic construct
CSGGSGARC

SEQ ID NO: 289 - protein, 9 aa, Artificial Sequence
  Peptide (1)..(9): synthetic construct
CPWWMMERC

(19) United States
(12) Patent Application Publication
(10) Pub. No.: US 2008/0176792 A1
(43) Pub. Date: Jul. 24, 2008

(54) VEGFR-1 TARGETING PEPTIDES

(76) Inventors: Wadih Arap, Houston, TX (US); Renata Pasqualini, Houston, TX (US); Ricardo J. Giordano, Sao Paulo (BR)

Correspondence Address: FULBRIGHT & JAWORSKI L.L.P., 600 CONGRESS AVE., SUITE 2400, AUSTIN, TX, 78701, US

(21) Appl. No.: 11/844,952

(22) Filed: Aug. 24, 2007

Related U.S. Application Data
(62) Division of application No. 10/363,205, filed on Jul. 15, 2004, now abandoned.
(60) Provisional application No. 60/231,266, filed on Sep. 8, 2000.

(51) Int. Cl. A61K 38/16; C07K 2/00; C07K 5/08; C07K 7/64; C07K 19/00; C12N 7/00; A61P 3/10; A61P 19/02; A61P 35/00; C12N 1/20; B32B 9/00; C12N 1/16; C12N 5/06

(52) U.S. Cl. 514/12; 530/300; 530/331; 530/321; 530/391.1; 435/235.1; 435/252.1; 428/402.2; 435/255.1; 435/375

(57) ABSTRACT

The present invention concerns novel methods of identifying peptide sequences that selectively bind to targets. In alternative embodiments, targets may comprise cells or clumps of cells, particles attached to chemicals compounds, molecules or aggregates, or parasites. In preferred embodiments, target cells are sorted before exposure to the phage library. The general method, Biopanning and Rapid Analysis of Selective Interactive Ligands (BRASIL) provides for rapid and efficient separation of phage that bind to targets, while preserving unbound phage. BRASIL may be used in preselection procedure to subtract phage that bind non-specifically to a first target before exposing the subtracted library to a second target. Certain embodiments concern targeting peptides identified by BRASIL and methods of use of such peptides for targeted delivery of therapeutic agents or imaging agents or diagnosis or treatment of diseases. Novel compositions comprising a first phase, second phase, target and a phage library are also disclosed.

This application claims the benefit of U.S. Provisional Patent Application No. 60/231,266 filed Sep. 8, 2000, and U.S. patent application Ser. No. 09/765,101, filed Jan. 17, 2001.

This invention was made with government support under grants DAMD 17-98-1-8041 and 17-98-1-8581 from the U.S. Army and grants 1R01CA78512-01A1, 1R01CA90810-01 and 1R01CA82976-01 from the National Institutes of Health. The government has certain rights in this invention.

### BACKGROUND OF THE INVENTION

1. Field of the Invention

The present invention concerns the fields of molecular medicine and targeted delivery. More specifically, the present invention relates to compositions and methods for identification and use of peptides that selectively target organs or tissues. In particular, the methods and compositions concern biopanning and rapid analysis of selective interactive ligands (BRASIL).

2. Description of Related Art

Therapeutic treatment of many human disease states is limited by the systemic toxicity of the therapeutic agents used. Cancer therapeutic agents in particular exhibit a very low therapeutic index, with rapidly growing normal tissues such as skin and bone marrow affected at concentrations of agent that are not much higher than the concentrations used to kill tumor cells. Treatment of cancer and other organ or tissue confined disease states would be greatly facilitated by the development of compositions and methods for targeted delivery to a desired organ or tissue of a therapeutic agent. Diagnostic imaging would also be facilitated by the targeted delivery of imaging agents to desired organs, tissues or diseased cells.

Recently, an in vivo selection system was developed using phage display libraries to identify organ or tissue targeting peptides in a mouse model system. Phage display libraries expressing transgenic peptides on the surface of bacteriophage were initially developed to map epitope binding sites of immunoglobulins (Smith and Scott, 1985, 1993). Such libraries can be generated by inserting random oligonucleotides into cDNAs encoding a phage surface protein, generating collections of phage particles displaying unique peptides in as many as 10⁹ permutations. (Pasqualini and Ruoslahti, 1996, Arap et al, 1998a; Arap et al 1998b).

Intravenous administration of phage display libraries to mice was followed by the recovery of phage from individual organs (Pasqualini and Ruoslahti, 1996). Phage were recovered that were capable of selective homing to the vascular beds of different mouse organs or tissues, based on the specific targeting peptide sequences expressed on the outer surface of the phage (Pasqualini and Ruoslahti, 1996). A variety of organ and tumor-homing peptides have been identified by this method (Rajotte et al., 1998, 1999; Koivunen et al., 1999; Burg et al., 1999; Pasqualini, 1999). Each of those targeting peptides bound to different receptors that were selectively expressed on the vasculature of the mouse target tissue (Pasqualini, 1999; Pasqualini et al., 2000; Folkman, 1995; Folkman 1997). Tumor-homing peptides bound to receptors that were upregulated in the tumor angiogenic vasculature of mice (Brooks et al., 1994b; Pasqualini et al., 2000). In addition to identifying individual targeting peptides selective for an organ or tissue (Pasqualini and Ruoslahti, 1996; Arap et al, 1998a; Koivunen et al., 1999), this system has been used to identify endothelial cell surface markers that are expressed in mice in vivo (Rajotte and Ruoslahti, 1999).

Attachment of therapeutic agents to targeting peptides resulted in the selective delivery of the agent to a desired organ or tissue in the mouse model system. Targeted delivery of chemotherapeutic agents and proapoptotic peptides to receptors located in tumor angiogenic vasculature resulted in a marked increase in therapeutic efficacy and a decrease in systemic toxicity in tumor-bearing mouse models (Arap et al., 1998a, 1998b; Ellerby et al., 1999).

This relative success notwithstanding, cell surface selection of phage libraries has been plagued by technical difficulties. A high number of non-binder and non-specific binder clones are recovered when phage libraries are incubated with cell suspensions or monolayers. Removal of this background phage binding by repeated washes is both labor-intensive and inefficient. Cells and potential ligands are frequently lost during the many washing steps required. Thus, there is a need for a rapid and efficient method for in vitro biopanning that retains the selectivity and specificity of in vivo methods, while providing decreased non-specific background.

Previous studies with phage display libraries have relied on a mouse model system to identify targeting peptides and their receptors, under the assumption that human targeting peptides are homologous. However, cell surface receptors may have a very different distribution and function in humans than in mice. Further, the mouse model system has been exploited to characterize targeting peptides for only a handful of specific organs. A need exists in the art for methods and compositions for identification of targeting sequences selective for human organs, tissues or cell types that can be of clinical use for targeted delivery of therapeutic agents

### SUMMARY OF THE INVENTION

The present invention solves a long-standing need in the art by providing compositions and in vitro methods for identifying targeting peptides that are selective for organs, tissues or cell types. In a preferred embodiment, such targeting peptides are identified by collecting samples of one or more organs, tissues, or cell types, separating the samples into isolated cells or small clumps of cells, suspending the cells or clumps in a first phase, exposing the cells or clumps of cells to a phage display library, layering the first phase over a second phase, and centrifuging the two phases so that the cells are pelleted at the bottom of a centrifuge tube. In a more preferred embodiment, the first phase is aqueous and the second phase is organic. In even more preferred embodiments, the cells are human cells. In certain embodiments, phage may be collected from the pellet by exposure to bacteria and phage clones may be plated, isolated and grown up in bulk culture. In alternative embodiments, phage inserts may be recovered from the pellet by PCR™ or other amplification techniques and the inserts sequenced to identify the targeting peptides. In certain embodiments, the organic phase comprises dibutylphtalate or a mixture of dibutylphthalate and cyclohexane. The methods disclosed herein are generally referred to herein as Biopanning and Rapid Analysis of Selective Interactive Ligands (BRASIL).

In alternative embodiments, the BRASIL method may be used to identify targeting peptides against virtually any chemical, molecule or complex of molecules. The separation of bound and unbound phage is preferably accomplished by partitioning bound phage from an aqueous phase into an organic phase. This requires that the target to which the phage bind be either denser than phage, larger than phage or preferably both. In preferred embodiments, the target is insoluble in the aqueous phase. In order to satisfy this requirement, chemicals, compounds, or molecules may be attached to a large insoluble particle, for example a glass, plastic, ceramic or magnetic bead. The skilled article will realize that the invention is not limited to beads and any large and/or dense particle may be used. The particle attached target may be exposed to a phage library in an aqueous phase and phage binding to the target partitioned into an organic phase. Although the examples shown herein illustrate the use of centrifugation to partition bound phage into the organic phase, the skilled artisan will realize that other types of partitioning may be used within the scope of the invention. For example, for targets attached to magnetic beads, a magnetic field could be imposed to pull the phage bound to beads into an organic phase.

In embodiments where cells are the targets, the cells may be mammalian cells, human cells, mouse cells or animal cells. Alternatively, cells may include any type of prokaryotic or eukaryotic cell, such as bacteria or unicellular microorganisms. In preferred embodiments, specific populations of cells may be prepared for use in BRASIL. For example, cells from leukemic patients may be sorted using a FACS (fluorescent activated cell sorter, Becton-Dickinson) to sort cancer cells from non-cancer cells. A phage library may be screened against cancerous cells only, either with or without a preselection subtraction against normal cells from the same patient. The skilled artisan will realize that cell sorting is not limited to leukemic samples, but rather may be practiced with any heterogenous population of cells.

In certain embodiments, targeting peptides identified by BRASIL are of use for the selective delivery of therapeutic agents, including but not limited to gene therapy vectors and fusion proteins, to specific organs, tissues or cell types in subjects. The skilled artisan will realize that the scope of the claimed methods of use include any disease state that can be treated by targeted delivery of a therapeutic agent to a desired organ, tissue or cell type.

The skilled artisan will understand that although the targeting peptides disclosed herein are particularly suited for use in human subjects, it is contemplated that they may be of use in other subjects such as mice, dogs, cats, horses, cows, sheep, pigs or any other mammal.

Certain embodiments concern targeting peptides identified by the BRASIL method. One embodiment of the present invention concerns isolated peptides of 100 amino acids or less in size, comprising at least 3 contiguous amino acids of a targeting peptide sequence, selected from SEQ ID NO:6, SEQ ID NO:7, SEQ ID NO:8, SEQ ID NO:11, any of SEQ ID NO:13 through SEQ ID NO:124 or any of SEQ ID NO:128 through SEQ ID NO:289.

In a preferred embodiment, the isolated peptide is 50 amino acids or less, more preferably 30 amino acids or less, more preferably 20 amino acids or less, more preferably 10 amino acids or less, or even more preferably 5 amino acids or less in size. In other preferred embodiments, the isolated peptide of claim 1 comprises at least 4, 5, 6, 7, 8, 9, 10, 11, 12, 13, 14, 15, 16, 17, 18, 19, 20, 21, 22, 23, 24 or 25 contiguous amino acids of a targeting peptide sequence, selected from SEQ ID NO:6, SEQ ID NO:7, SEQ ID NO:8, SEQ ID NO:11, any of SEQ ID NO:13 through SEQ ID NO:124 or any of SEQ ID NO:128 through SEQ ID NO:289.

In certain embodiments, the isolated peptide is attached to a molecule. In preferred embodiments, the attachment is a covalent attachment. In additional embodiments, the molecule is a drug, a chemotherapeutic agent, a radioisotope, a pro-apoptosis agent, an anti-angiogenic agent, a hormone, a cytokine, a growth factor, a cytotoxic agent, a peptide, a protein, an antibiotic, an antibody, a Fab fragment of an antibody, an imaging agent, a nucleic acid or an antigen. Those molecules are representative only. Molecules within the scope of the present invention include virtually any molecule that may be attached to a targeting peptide and administered to a subject. In preferred embodiments, the pro-apoptosis agent is gramicidin, magainin, mellitin, defensin, cecropin, (KLAKLAK)₂ (SEQ ID NO:1), (KLAKKLA)₂ (SEQ ID NO:2), (KAAKKAA)₂ (SEQ ID NO:3) or (KLGKKLG)₃ (SEQ ID NO:4). In other preferred embodiments, the anti-angiogenic agent is thrombospondin, angiostatin, endostatin or pigment epithelium-derived factor. In further preferred embodiments, the cytokine is interleukin 1 (IL-1), IL-2, IL-5, IL-10, IL-11, IL-12, IL-18, interferon-γ (IF-γ), IF-α, IF-β, tumor necrosis factor-α (TNF-α), or GM-CSF (granulocyte macrophage colony stimulating factor). Such examples are representative only and are not intended to exclude other pro-apoptosis agents, anti-angiogenic agents or cytokines known in the art.

In other embodiments, the isolated peptide is attached to a macromolecular complex. In preferred embodiments, the attachment is a covalent attachment. In other preferred embodiments, the macromolecular complex is a virus, a bacteriophage, a bacterium, a liposome, a microparticle, a magnetic bead, a cell or a microdevice. These are representative examples only. Macromolecular complexes within the scope of the present invention include virtually any macromolecular complex that may be attached to a targeting peptide and administered to a subject. In other preferred embodiments, the isolated peptide is attached to a eukaryotic expression vector, more preferably a gene therapy vector.

In another embodiment, the isolated peptide is attached to a solid support, preferably magnetic beads, Sepharose beads, agarose beads, a nitrocellulose membrane, a nylon membrane, a column chromatography matrix, a high performance liquid chromatography (HPLC) matrix or a fast performance liquid chromatography (FPLC) matrix. Such attached peptides may be of use, for example, to purify or isolate an antibody, protein, peptide or other ligand that binds to the targeting peptide. In certain embodiments, this binding may be used to identify endogenous receptors, ligands or receptor:ligand pairs that are mimicked by the targeting peptide.

Additional embodiments of the present invention concern fusion proteins comprising at least 3 contiguous amino acids of a sequence selected from SEQ ID NO:61 SEQ ID NO:7, SEQ ID NO:8, SEQ ID NO:11, any of SEQ ID NO:13 through SEQ ID NO:124 or any of SEQ ID NO:128 through SEQ ID NO:289.

Certain other embodiments concern compositions comprising the claimed isolated peptides or fusion proteins in a pharmaceutically acceptable carrier. Further embodiments concern kits comprising the claimed isolated peptides or fusion proteins in one or more containers.

Additional embodiments concern kits comprising compositions and apparatus for performing BRASIL. Kit components may include, but are not limited to, any composition or apparatus that may be of use in performing BRASIL, such as solutions, buffers, media, organic phase, bacteria, phage libraries, control phage, centrifugation tubes, etc.

Other embodiments concern methods of targeted delivery comprising selecting a targeting peptide for a desired organ or tissue, attaching said targeting peptide to a molecule, macromolecular complex or gene therapy vector, and providing said peptide attached to said molecule, complex or vector to a subject. Preferably, the targeting peptide is selected to include at least 3 contiguous amino acids from SEQ ID NO:6, SEQ ID NO:7, SEQ ID NO:8, SEQ ID NO:11, any of SEQ ID NO:13 through SEQ ID NO:124 or any of SEQ ID NO:128 through SEQ ID NO:289. In certain preferred embodiments, the molecule attached to the targeting peptide is a chemotherapeutic agent, an antigen or an imaging agent. The skilled artisan will realize that within the scope of the present invention any organ, tissue or cell type can be targeted for delivery, using targeting peptides attached to any molecule, macromolecular complex or gene therapy vector.

Certain embodiments of the present invention concern methods for imaging an organ, tissue, or cell type comprising selecting a peptide targeted to said organ or tissue, attaching an imaging agent to said peptide, administering said peptide to a subject and obtaining an image. In preferred embodiments, the targeted cells are associated with a disease or other condition. In other preferred embodiments, the targeting peptide comprises at least three contiguous amino acids selected from SEQ ID NO:6, SEQ ID NO:7, SEQ ID NO:8, SEQ ID NO:11, any of SEQ ID NO:13 through SEQ ID NO:124 or any of SEQ ID NO:128 through SEQ ID NO:289.

In other embodiments, the present invention concerns methods of diagnosing a disease state, comprising selecting a peptide targeted to cells associated with such disease state, attaching an imaging agent to said peptide, administering said peptide and imaging agent to a subject suspected of having the disease, and diagnosing the presence or absence of the disease based on the distribution of said peptide and imaging agent within said subject. Preferably, the targeting peptide contains at least 3 contiguous amino acids selected from SEQ ID NO:6, SEQ ID NO:7, SEQ ID NO:8, SEQ ID NO:11, any of SEQ ID NO:13 through SEQ ID NO:124 or any of SEQ ID NO:128 through SEQ ID NO:289. In preferred embodiments, the disease state is diabetes mellitus, inflammatory disease, rheumatoid arthritis, atherosclerosis, cancer, autoimmune disease, bacterial infection or viral infection. In a more preferred embodiment, the disease state is metastatic cancer.

Additional embodiments concern methods for identifying a receptor for a targeting peptide, comprising contacting said peptide to an organ, tissue or cell containing said receptor, allowing said peptide to bind to said receptor, and identifying said receptor by its binding to said peptide. In preferred embodiments, the targeting peptide contains at least three contiguous amino acids selected from SEQ ID NO:6, SEQ ID NO:7, SEQ ID NO:8, SEQ ID NO:11, any of SEQ ID NO:13 through SEQ ID NO:124 or any of SEQ ID NO:128 through SEQ ID NO:289. The skilled artisan will realize that the contacting step can utilize samples of organs, tissues or cells, or may alternatively utilize homogenates or detergent extracts of the organs, tissues or cells. In certain embodiments, the cells to be contacted may be genetically engineered to express a suspected receptor for the targeting peptide. In a preferred embodiment, the targeting peptide is modified with a reactive moiety that allows its covalent attachment to said receptor. In a more preferred embodiment, the reactive moiety is a photoreactive group that becomes covalently attached to the receptor when activated by light. In another preferred embodiment, the peptide is attached to a solid support and the receptor is purified by affinity chromatography. In other preferred embodiments, the solid support comprises magnetic beads, Sepharose beads, agarose beads, a nitrocellulose membrane, a nylon membrane, a column chromatography matrix, a high performance liquid chromatography (HPLC) matrix or a fast performance liquid chromatography (FPLC) matrix. In certain embodiments, the targeting peptide inhibits the activity of the receptor upon binding to the receptor. The skilled artisan will realize that receptor activity can be assayed by a variety of methods known in the art, including but not limited to catalytic activity and binding activity. In another preferred embodiment, the receptor is an endostatin receptor, a metalloprotease or an aminopeptidase.

Other embodiments of the present invention concern isolated nucleic acids of 300 nucleotides or less in size, encoding a targeting peptide. In preferred embodiments, the isolated nucleic acid is 250, 225, 200, 175, 150, 125, 100, 75, 50, 40, 30, 20 or even 10 nucleotides or less in size. In other preferred embodiments, the isolated nucleic acid is incorporated into a eukaryotic or a prokaryotic expression vector. In even more preferred embodiments, the vector is a plasmid, a cosmid, a yeast artificial chromosome (YAC), a bacterial artificial chromosome (BAC), a virus or a bacteriophage. In other preferred embodiments, the isolated nucleic acid is operatively linked to a leader sequence that localizes the expressed peptide to the extracellular surface of a host cell.

Additional embodiments of the present invention concern methods of treating a disease state comprising selecting a targeting peptide that targets cells associated with the disease state, attaching one or more molecules effective to treat the disease state to the peptide, and administering the peptide to a subject with the disease state. Preferably, the targeting peptide includes at least three contiguous amino acids selected from SEQ ID NO:6, SEQ ID NO:7, SEQ ID NO:8, SEQ ID NO:11, any of SEQ ID NO:13 through SEQ ID NO:124 or any of SEQ ID NO:128 through SEQ ID NO:289. In preferred embodiments the disease state is diabetes mellitus, inflammatory disease, rheumatoid arthritis, atherosclerosis, cancer, autoimmune disease, bacterial infection and viral infection.

Another embodiment of the present invention concerns molecular adaptors for targeted gene therapy. In a preferred embodiment, the molecular adaptor comprises a Fab fragment of an antibody that is specific for a gene therapy vector, covalently attached to a targeting peptide sequence that provides selective targeting to a desired organ or tissue. The skilled artisan will realize that the present invention may include any gene therapy vector that is known in the art. The vector binding portion of the molecular adaptor is not limited to Fab fragments of antibodies, but may include any other molecule that can be used to attach a targeting peptide to a gene therapy vector. The only requirement is that the gene therapy vector should be selectively targeted to a desired organ or tissue in the presence of the molecular adaptor.

Another embodiment of the present invention concerns compositions and methods of use of tumor targeting peptides against cancers. Tumor targeting peptides identified by the methods disclosed in the instant application may be attached to therapeutic agents, including but not limited to molecules or macromolecular assemblages and administered to a subject with cancer, providing for increased efficacy and decreased systemic toxicity of the therapeutic agent. Therapeutic agents within the scope of the present invention include but are not limited to chemotherapeutic agents, radioisotopes, pro-apoptosis agents, cytotoxic agents, cytostatic agents and gene therapy vectors. Targeted delivery of such therapeutic agents to tumors provides a significant improvement over the prior art for increasing the delivery of the agent to the tumor, while decreasing the inadvertent delivery of the agent to normal organs and tissues of the subject. In a preferred embodiment, the tumor targeting peptide is incorporated into the capsule of a phage gene therapy vector to target delivery of the phage to angiogenic endothelial cells in tumor blood vessels.

A further embodiment of the present invention concerns methods for identifying new tumor targeting peptides, using phage display libraries that incorporate reporter genes. Administration of the reporter gene phage library to a subject with a tumor is followed by recovery of phage from the tumor and identification of tumor targeting peptides by sequencing selected portions of the phage genome that contain the nucleic acid sequence encoding the targeting peptide. While these embodiments of the present invention concern tumors, the skilled artisan will realize that within the scope of the present invention other disease states that are localized to specific organs or tissues may also be treated with enhanced therapeutic efficacy and decreased systemic toxicity using the methods and compositions disclosed herein.

Yet another embodiment of the present invention concerns methods of identifying targeting peptides against antibodies from a subject with a disease state, comprising obtaining a sample of serum from the subject, obtaining antibodies from the sample, adding a phage display library to the antibodies and collecting phage bound to the antibodies. In preferred embodiments, the antibodies are attached to a solid support, more preferably attached to protein G attached to beads. In another preferred embodiment, a subtraction step is added where the phage display library is first screened against antibodies from a subject who does not have the disease state. Only phage that do not bind to these control antibodies are used to obtain phage binding to the diseased subject's antibodies.

In other preferred embodiments, phage that bind to a target organ or tissue, for example to placenta, may be pre-screened or post-screened against a subject lacking that organ or tissue. Phage that bind to the subject lacking the target organ or tissue are removed from the library.

Other embodiments concern methods of obtaining antibodies against an antigen. In preferred embodiments, the antigen comprises one or more targeting peptides. The targeting peptides are prepared and immobilized on a solid support, a sample containing antibodies is added and antibodies that bind to the targeting peptides are collected.

In other preferred embodiments, a phage display library displaying the antigen binding portions of antibodies from a subject is prepared, the library is screened against one or more antigens and phage that bind to the antigens are collected. In more preferred embodiments, the antigen is a targeting peptide.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. BRASIL principle. A suspension of single cells or small clumps of cells that has been incubated with phage (library or single clones) in an upper first phase is centrifuged over a preferably non-miscible oil lower second phase. Because the second phase has an intermediate specific density, upon optimized centrifugation conditions, the cells will enter the lower phase and pellet at the bottom of the tube, carrying with them only the specifically bound phage. The remaining unbound phage stay in the upper phase. The cell pellet is then carefully recovered. Targeting peptide sequences may be identified by amplification and sequencing of the phage inserts, either with or without recovery of the phage by infection of a host E. coli.

FIG. 2A. BRASIL method optimization. A single-cell suspension of Kaposi sarcoma-derived cells (KS1767) was incubated with increasing titers of a phage displaying an alpha v integrin-binding motif (RGD-4C phage) or a control phage with no peptide insert (Fd phage). KS1767 cells were chosen because they express high levels of alpha v integrins. Cells and phage were incubated for 4 hours on ice (to prevent phage internalization) and centrifuged for 10 minutes at 10,000 g. The phage bound to the KS1767 cells were recovered by infection of a host E. coli, and plated in LB/tet agar plates at 37° C. overnight. Finally, the phage transducing units were counted. Extremely low backgrounds were observed. Under non-saturated conditions, a conservative mean estimate of the enrichment of RGD-4C phage in relation to Fd phage is 500-1000. This experiment was repeated three times with similar results. Mean and standard deviation are shown.

FIG. 2B. The synthetic RGD-4C peptide—but not the RGE control peptide—in solution inhibited 99.99% of the RGD-4C phage binding to KS1767 cells

FIG. 3. Pre-clearing protocol using BRASIL to selectively remove phage from a phage display library.

FIG. 4A. Binding of phage clone-19 to immobilized VEGF receptors. Clone-19 (black bars) binds to VEGF-R1 and NRP-1 but not VEGF-R2 or BSA. No binding of insertless Fd phage could be detected (hash bars).

FIG. 4B. The binding to the VEGF-R1 (black circle) and NRP-1 (open square) could be completely blocked by 10-20 ng/ml of VEGF₁₆₅.

FIG. 5. HUVEC cells were cultured in 24-well plates and starved for 24 with basal medium without any sera and supplements. Phage clone-19 or RGD.4C (which binds to HUVEC) were added at 10¹⁰ T.U. per well. VEGF₁₆₅ (20 ng/ml) or basal (starved) medium were added as positive and negative controls. Cell proliferation was measured by the MMT method. Clone-19 promoted cell proliferation comparably to the positive control (VEGF-165). The RGD.4C peptide, which also binds to HUVEC, resulted in a cell proliferation rate only slightly above the negative control.

FIG. 6A-6C. Binding of selected phage clones to a subconfluent human urothelial cell monolayer. Insertless fd-tet phage were included as negative control. Results are means of triplicate wells relative to binding of fd-tet phage, that was set as 1. Input of phage was 1×10⁸ T.U. per well. Bound phage were detected after intensive washing by infection with log phase K91 bacteria and plating of serial dilutions.

FIG. 7. Binding of selected phage clones to the human breast cancer cell line MDA-MBA435 (white bars) as well as the urothelial tumor cell lines T24 (light grey bars) and RT4 (dark grey bars). Insertless fd-tet phage were included as controls. Results are given as mean of triplicate wells relative to binding of fd-tet phage, that was set as 1. Input of phage was 1×10⁸ T.U. per well. Bound phage were detected after intensive washing by infection with log phase K91 bacteria and plating of serial dilutions.

FIG. 8. Inhibition of VHALES (SEQ ID NO:25) phage binding to RT4 tumor cells was inhibited by synthetic VHALE (SEQ ID NO:25) (black squares) but not by the control peptide CARAC (SEQ ID NO:5) (white squares). Binding of VHALES (SEQ ID NO:25) phage was 5.4 fold higher than insertless fd-tet phage. A subconfluent monolayer of RT4 tumor cells was incubated with 1×10⁸ T.U. of VHALES (SEQ ID NO:25) phage per well in presence of increasing amounts of VHALES (SEQ ID NO:25) and control peptide. Results are given as mean of triplicate wells. Bound phage were detected after intensive washing by infection with log phase K91 bacteria and plating of serial dilutions.

FIG. 9. Binding of selected phage clones to porcine urothelium in a dot blot chamber assay. Three wells were pooled as one field, and results represent the mean of triplicate fields relative to binding of insertless fd-tet phage, that was set as 1. Input of phage was 1×10⁸ T.U. per well. Bound phage were detected after intensive washing by infection with log phase K91 bacteria and plating of serial dilutions.

FIG. 10. Influence of the GAG-layer on phage binding. In the dot blot chamber assay portions of a porcine bladder mucosa were treated with 0.1M HCl for 2 min prior to remove the GAG-layer. Binding to treated surface is given relative to untreated surface, that was set as 1. Bound phage were detected after intensive washing by infection with log phase K91 bacteria and plating of serial dilutions.

FIG. 11. Binding of selected clones to human bone marrow cells by BRASIL.

### DESCRIPTION OF ILLUSTRATIVE EMBODIMENTS

As used herein in the specification, “a” or “an” may mean one or more. As used herein in the claim(s) in conjunction with the word “comprising” the words “a” or “an” may mean one or more than one. As used herein “another” may mean at least a second or more of an item.

A “targeting peptide” is a peptide comprising a contiguous sequence of amino acids that is characterized by selective localization to a target organ, tissue or cell type, preferably of human origin. Selective localization may be determined, for example, by methods disclosed below, wherein the putative targeting peptide sequence is incorporated into a protein that is displayed on the outer surface of a phage. In certain embodiments, targeting phage that have been identified by BRASIL are administered to a subject followed by collection of one or more organs, tissues or cell types from the subject and identification of phage found in the target organ, tissue or cell type. A phage expressing a targeting peptide sequence is considered to be selectively localized if it exhibits greater binding in the target compared to a control tissue, organ or cell type. Another alternative method of determining selective localization is that phage expressing the putative target peptide exhibit at least a two-fold, more preferably at least a three-fold enrichment in the target compared to control phage that express a non-specific peptide or that have not been genetically engineered to express any putative target peptides. Another method to determine selective localization is that localization to the target of phage expressing the target peptide is at least partially blocked by the co-administration of a synthetic peptide containing the target peptide sequence. “Targeting peptide” and “homing peptide” are used synonymously herein.

A “phage display library” means a collection of phage that have been genetically engineered to express a set of putative targeting peptides on their outer surface. In preferred embodiments, DNA sequences encoding the putative targeting peptides are inserted in frame into a gene encoding a phage capsule protein. In other preferred embodiments, the putative targeting peptide sequences are in part random mixtures of all twenty amino acids and in part non-random. In certain preferred embodiments the putative targeting peptides of the phage display library exhibit one or more cysteine residues at fixed locations within the targeting peptide sequence.

A “macromolecular complex” refers to a collection of molecules that may be random, ordered or partially ordered in their arrangement. The term encompasses biological organisms such as bacteriophage, viruses, bacteria, unicellular pathogenic organisms, multicellular pathogenic organisms and prokaryotic or eukaryotic cells. The term also encompasses non-living assemblages of molecules, such as liposomes, microcapsules, microparticles, microdevices and magnetic beads. The only requirement is that the complex contains more than one molecule. The molecules may be identical, or may differ from each other.

A “receptor” for a targeting peptide includes but is not limited to any molecule or complex of molecules that binds to a targeting peptide. Non-limiting examples of receptors include peptides, proteins, glycoproteins, lipoproteins, epitopes, lipids, carbohydrates, multi-molecular structures, a specific conformation of one or more molecules and a morphoanatomic entity. In preferred embodiments, a “receptor” is a naturally occurring molecule or complex of molecules that is present on the lumenal surface of cells forming blood vessels within a target organ or tissue.

A “subject” refers generally to a mammal. In certain preferred embodiments, the subject is a mouse or rabbit. In even more preferred embodiments, the subject is a human.

### Phage Display

The methods described herein for identification of targeting peptides involve the in vitro administration of phage display libraries. Various methods of phage display and methods for producing diverse populations of peptides are well known in the art. For example, U.S. Pat. Nos. 5,223,409; 5,622,699 and 6,068,829, each of which is incorporated herein by reference, describe methods for preparing a phage library. The phage display technique involves genetically manipulating bacteriophage so that small peptides can be expressed on their surface (Smith and Scott, 1985, 1993). The potential range of applications for this technique is quite broad, and the past decade has seen considerable progress in the construction of phage-displayed peptide libraries and in the development of screening methods in which the libraries are used to isolate peptide ligands. For example, the use of peptide libraries has made it possible to characterize interacting sites and receptor-ligand binding motifs within many proteins, such as antibodies involved in inflammatory reactions or integrins that mediate cellular adherence. This method has also been used to identify novel peptide ligands that serve as leads to the development of peptidomimetic drugs or imaging agents (Arap et al., 1998a). In addition to peptides, larger protein domains such as single-chain antibodies can also be displayed on the surface of phage particles (Arap et al., 1998a).

Previously, amino acid sequences for targeting a given organ or tissue have been isolated by in vivo “biopanning” Pasqualini and Ruoslahti, 1996; Pasqualini, 1999). In brief, a library of phage containing putative targeting peptides is administered to an animal or human subject and samples of organs or tissues containing phage are collected. In examples utilizing filamentous phage, the phage may be propagated in vitro between rounds of biopanning in pilus-positive bacteria. The bacteria are not lysed by the phage but rather secrete multiple of copies of phage that display a particular insert. Phage that bind to a target molecule can be eluted from the target organ or tissue and then amplified by growing them in host bacteria. The amplified phage may be administered to a second subject and samples of organs or tissues again collected. Multiple rounds of biopanning may be performed until a population of selective binders is obtained. The amino acid sequence of the peptides is determined by sequencing the DNA corresponding to the targeting peptide insert in the phage genome. The identified targeting peptide can then be produced as a synthetic peptide by standard protein chemistry techniques (Arap et al., 1998a, Smith et al., 1985). This approach allows circulating targeting peptides to be detected in an unbiased functional assay, without any preconceived notions about the nature of their target. Once a candidate target is identified as the receptor of a targeting peptide, it can be isolated, purified and cloned by using standard biochemical methods (Pasqualini, 1999; Rajotte and Ruoslahti, 1999).

The in vitro methods disclosed herein also use phage display libraries. However, rather than injecting the library into a live host, samples of target organs, tissues or cell types are exposed to the phage display library in vitro.

Choice of Phage Display System.

In vivo selection studies performed in mice preferentially employed libraries of random peptides expressed as fusion proteins with the gene III capsule protein in the fUSE5 vector (Pasqualini and Ruoslahti, 1996). The number and diversity of individual clones present in a given library is a significant factor for the success of in vivo selection. Primary libraries are preferred, which are less likely to have an over-representation of defective phage clones (Koivunen et al., 1999). The preparation of a library may be optimized to between 10⁸-10⁹ transducing units (T.U.)/ml. A bulk amplification strategy may be applied between rounds of selection.

Phage libraries displaying linear, cyclic, or double cyclic peptides may be used. However, phage libraries displaying 3 to 10 random residues in a cyclic insert (CX₃₋₁₀C) are preferred, since single cyclic peptides tend to have a higher affinity for the target organ than linear peptides. Libraries displaying double-cyclic peptides (such as CX₃C X₃C X₃C; Rojotte et al., 1998) have been successfully used. However, the production of the cognate synthetic peptides, although possible, can be complex due to the multiple conformers with different disulfide bridge arrangements.

Identification of Homing Peptides and Receptors by in Vivo Phage Display in Mice.

In vivo selection of peptides from phage-display peptide libraries administered to mice has been used to identify targeting peptides selective for normal mouse brain, kidney, lung, skin, pancreas, retina, intestine, uterus, prostate, and adrenal gland (Pasqualini and Ruoslahti, 1996; Pasqualini, 1999; Rajotte et al., 1998). These results show that the vascular endothelium of normal organs is sufficiently heterogenous to allow differential targeting with peptide probes (Pasqualini and Ruoslahti, 1996; Rajotte et al., 1998). A panel of peptide motifs that target the blood vessels of tumor xenografts in nude mice has been assembled (Arap et al., 1998a; reviewed in Pasqualini, 1999). These motifs include the RGD-4C, NGR, and GSL peptides. The RGD-4C peptide has previously been identified as selectively binding αv integrins and has been shown to home to the vasculature of tumor xenografts in nude mice (Arap et al., 1998a, 1998b; Pasqualini et al., 1997).

The receptors for the tumor homing RGD4C targeting peptide have been identified as αv integrins (Pasqualini et al., 1997). The αv integrins play an important role in angiogenesis. The αvβ3 and αvβ5 integrins are absent or expressed at low levels in normal endothelial cells but are induced in angiogenic vasculature of tumors (Brooks et al., 1994; Hammes et al., 1996). Aminopeptidase N/CD13 has recently been identified as an angiogenic receptor for the NGR motif (Burg et al., 1999). Aminopeptidase N/CD13 is strongly expressed not only in the angiogenic blood vessels of prostate cancer in TRAMP mice but also in the normal epithelial prostate tissue.

Tumor-homing phage co-localize with their receptors in the angiogenic vasculature of tumors but not in non-angiogenic blood vessels in normal tissues (Arap et al., 1998b). Immunohistochemical evidence shows that vascular targeting phage bind to human tumor blood vessels in tissue sections (Pasqualini et al., 2000) but not to normal blood vessels. A negative control phage with no insert (fd phage) did not bind to normal or tumor tissue sections. The expression of the angiogenic receptors was evaluated in cell lines, in non-proliferating blood vessels and in activated blood vessels of tumors and other angiogenic tissues such as corpus luteum. Flow cytometry and immunohistochemistry showed that these receptors are expressed in a number of tumor cells and in activated HUVECs (data not shown). The angiogenic receptors were not detected in the vasculature of normal organs of mouse or human tissues.

The distribution of these receptors was analyzed by immunohistochemistry in tumor cells, tumor vasculature, and normal vasculature. Alpha v integrins, CD13, aminopeptidase A, NG2, and MMP-2/MM-9—the known receptors in tumor blood vessels—are specifically expressed in angiogenic endothelial cells and pericytes of both human and murine origin. Angiogenic neovasculature expresses markers that are either expressed at very low levels or not at all in non-proliferating endothelial cells (not shown).

The markers of angiogenic endothelium include receptors for vascular growth factors, such as specific subtypes of VEGF and basic FGF receptors, and αv integrins, among many others (Mustonen and Alitalo, 1995). Thus far, identification and isolation of novel molecules characteristic of angiogenic vasculature has been slow, mainly because endothelial cells undergo dramatic phenotypic changes when grown in culture (Watson et al., 1995).

Many of these tumor vascular markers are proteases and some of the markers also serve as viral receptors. Alpha v integrins are receptors for adenoviruses (Wickham et al., 1997c) and CD13 is a receptor for coronaviruses (Look et al., 1989). MMP-2 and MMP-9 are receptors for echoviruses (Koivunen et al., 1999). Aminopeptidase A also appears to be a viral receptor. Bacteriophage may use the same cellular receptors as eukaryotic viruses. These findings suggest that receptors isolated by phage display will have cell internalization capability, a key feature for utilizing the identified peptide motifs as targeted gene therapy carriers.

Targeted Delivery

Peptides that home to tumor vasculature have been coupled to cytotoxic drugs or proapoptotic peptides to yield compounds that were more effective and less toxic than the parental compounds in experimental models of mice bearing tumor xenografts (Arap et al., 1998a; Ellerby et al, 1999). The insertion of the RGD-4C peptide into a surface protein of an adenovirus has produced an adenoviral vector that may be used for tumor targeted gene therapy (Arap et al., 1998b).

### BRASIL

In certain embodiments, separation of phage bound to the cells of a target organ or tissue from unbound phage is achieved using the BRASIL technique. In BRASIL (Biopanning and Rapid Analysis of Selective Interactive Ligands), an organ or tissue is gently separated into cells or small clumps of cells that are suspended in an first phase. The first phase is layered over a second phase of appropriate density and centrifuged. Cells attached to bound phage are pelleted at the bottom of the centrifuge tube, while unbound phage remain in the first phase. This allows a more efficient separation of bound from unbound phage, while maintaining the binding interaction between phage and cell. BRASIL may be performed by an in vitro protocol, where the cells are exposed to the phage library in the aqueous phase before centrifugation. In preferred embodiments, the first phase is aqueous and the second phase is organic. Specific non-limiting examples of organic phases that may be employed within the scope of the present invention are disclosed below.

Although the cells shown in the Examples below are primarily human cells, the invention is not limiting for the type of cell that may be used. Virtually any type of prokaryotic or eukaryotic cell may be used with BRASIL, including but not limited to human, mouse, mammalian, animal or plant cells, bacteria and unicellular organisms such as amoeba, spores, yeast, molds, algae, Giardia or dinoflagellates. In certain embodiments, the cells to be screened by BRASIL may first be sorted, for example using an FACS apparatus (Becton Dickinson) to separate heterogenous populations of cells into homogenous populations of cells.

In alternative embodiments, the target used to screen the phage library may include non-cellular targets, such as chemicals, compounds, molecules or aggregates of molecules. Target molecules of potential use in BRASIL include but are not limited to proteins, proteoglycans, carbohydrates, lipids, glycolipids, sphingolipids and lipoproteins. In preferred embodiments, such non-cellular targets may be attached either covalently or non-covalently to a larger particle, such as a glass, plastic, ceramic or magnetic bead. Linkers may be used for the attachment to increase the accessibility of the target to the phage targeting peptides. In such embodiments, the skilled artisan will realize that other methods of separating bound phage into an organic phase may be used besides centrifugation. For example, where magnetic particles are used, the particles may be partitioned into the organic phase by imposition of a magnetic field. If the particle is sufficiently large or dense, settling of the particle under the influence of gravity may be used to partition the bound phage into the organic phase. The invention is not limiting to the method of partitioning bound phage into an organic phase and any method known in the art for separating phage bound to particles or cells into an organic or other second phase may be used within the scope of the invention.

The invention is not limiting as to the exact composition of the first and second phases, as long as the cells to be pelleted have a density that is higher than that of the second phase, and the second phase has a density that is higher than the first phase.

In preferred embodiments, the second phase has a density of about 1.02 to 1.04, while the first phase has a density of about 1.00. The cells or clumps of cells used for BRASIL preferably have a density of greater than 1.04 gm/ml. The skilled artisan will realize that specific cell types may vary in density and that optimization of BRASIL by adjustment of phase density may be appropriate. In preferred embodiments, in order to prevent mixing and dilution, the first and second phases are immiscible. However, step gradient centrifugation using miscible phases is known in the art and may be used in the practice of the present invention, for example using cesium chloride, sucrose, PEG (polyethylene glycol), Ficoll or Percoll solutions of appropriate density.

A variety of organic solvents of known density are available for use. Non-limiting examples of organic solvents with reported densities between 1.02 and 1.04 include diisoamyl phthalate (1.021), phenyl butyrate (1.038), tributyrin (1.035), 9-ethylanthracene (1.041), methyl-diphenylamine (1.048), 1-2-dimethoxy-4-(2-propyl)-benzene (1.039), alpha-phenyl-benzenethanol (1.036), 3-methyl-benzenthiol (1.041), acetaldehyde semicarbazone (1.030), phenylacetaldehyde (1.027) and dibenzylamine (1.026). Other organic compounds and their densities may be found, for example, in the Handbook of Chemistry and Physics, 50^{th} Edition, pp. C-75 to C-541, the Chemical Rubber Co., Cleveland, Ohio 1969. Of course, it is not necessary that the organic phase be comprised of a single organic solvent and it is contemplated within the scope of the invention that an organic phase of appropriate density may be produced by mixing organic solvents of different densities, as disclosed in the Examples below. Additional mixtures may be designed using routine techniques known in the art. The skilled artisan will realize that densities often are temperature dependent and that the appropriate densities are obtained at the temperature of the centrifuge used to pellet the cells. In various embodiments, that temperature may range from room temperature to about 4° C. For purposes of centrifugation, any organic phase utilized should be a liquid at the temperature used.

The artisan will further realize that optimization of second phase density may be required for different cell types. For example, different densities are observed for rat hepatocytes (1.07-1.10), Kupffer cells (1.05-1.06), human thrombocytes (1.04-1.06), lymphocytes (1.06-1.08), granulocytes (1.08-1.09), erythrocytes (1.09-1.10) and E. coli (1.13). All of these cell types would be expected to pellet through a second organic phase of about 1.03 density. It is further realized that the osmolarity of the first (aqueous) phase may affect the density of cells, particularly cells that are not bound by a rigid cell wall. In preferred embodiments, the osmolarity of the medium is approximately equal to the osmolarity of cells in situ (approximately 150 mM salt concentration). A wide variety of media of physiological osmolarity are known in the art, such as phosphate or Tris buffered saline (PBS or TBS).

The skilled artisan will also realize that organic phases of high toxicity are to be avoided. For example, organic solvents such as phenol or formaldehyde that result in denaturation of proteins are undesirable for use as a second phase. The toxicity properties of organic solvents are well known in the art.

In certain embodiments, a subtraction protocol is used with BRASIL to further reduce background phage binding. The purpose of subtraction is to remove phage from the library that bind to cells other than the cell of interest, or that bind to inactivated cells. In alternative embodiments, the phage library may be screened against a control cell line, tissue or organ sample that is not the targeted cell, tissue or organ. After subtraction the library may be screened against the cell, tissue or organ of interest. In another alternative embodiment, an unstimulated, quiescent cell line, tissue or organ may be screened against the library and binding phage removed. The cell line, tissue or organ is then activated, for example by administration of a hormone, growth factor, cytokine or chemokine and the activated cell line screened against the subtracted phage library.

Other methods of subtraction protocols are known and may be used in the practice of the present invention, for example as disclosed in U.S. Pat. Nos. 5,840,841, 5,705,610, 5,670,312 and 5,492,807, incorporated herein by reference.

### Proteins and Peptides

In certain embodiments, the present invention concerns novel compositions comprising at least one protein or peptide. As used herein, a protein or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide” and “peptide are used interchangeably herein.

In certain embodiments the size of the at least one protein or peptide may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues.

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid, including but not limited to those shown on Table 1 below.

TABLE 1
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino- | AHyl | allo-Hydroxylysine |
| | propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for known genes may be amplified and/or expressed using the techniques disclosed herein or as would be know to those of ordinary skill in the art. Alternatively, various commercial preparations of proteins, polypeptides and peptides are known to those of skill in the art.

Peptide Mimetics

Another embodiment for the preparation of polypeptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993), incorporated herein by reference. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting peptides disclosed herein, but with altered and even improved characteristics.

Fusion Proteins

Other embodiments of the present invention concern fusion proteins. These molecules generally have all or a substantial portion of a targeting peptide, linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In preferred embodiments, the fusion proteins of the instant invention comprise a targeting peptide linked to a therapeutic protein or peptide. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. These examples are not meant to be limiting and it is contemplated that within the scope of the present invention virtually any protein or peptide could be incorporated into a fusion protein comprising a targeting peptide. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

Protein Purification

In certain embodiments a protein or peptide may be isolated or purified. Protein purification techniques are well known to those of skill in the art. These techniques involve, at one level, the homogenization and crude fractionation of the cells, tissue or organ to polypeptide and non-polypeptide fractions. The protein or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. An example of receptor protein purification by affinity chromatography is disclosed in U.S. Pat. No. 5,206,347, the entire text of which is incorporated herein by reference. A particularly efficient method of purifying peptides is fast protein liquid chromatography (FPLC) or even HPLC.

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind to. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. In various embodiments, affinity chromatography may be performed to purify a targeting peptide, an antibody against a targeting peptide, an antigen that binds to an antibody, an endogenous receptor for a targeting peptide, or a ligand for a targeting peptide.

Synthetic Peptides

Because of their relatively small size, the targeting peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, usually from about 6 up to about 35 to 50 amino acids, can be readily synthesized by such methods. Alternatively, recombinant DNA technology may be employed wherein a nucleotide sequence which encodes a peptide of the invention is inserted into an expression vector, transformed or transfected into an appropriate host cell, and cultivated under conditions suitable for expression.

Antibodies

In certain embodiments, it may be desirable to make antibodies against the identified targeting peptides or their receptors. The appropriate targeting peptide or receptor, or portions thereof, may be coupled, bonded, bound, conjugated, or chemically-linked to one or more agents via linkers, polylinkers, or derivatized amino acids. This may be performed such that a bispecific or multivalent composition or vaccine is produced. It is further envisioned that the methods used in the preparation of these compositions are familiar to those of skill in the art and should be suitable for administration to human subjects, i.e., pharmaceutically acceptable. Preferred agents are the carriers are keyhole limpet hemocyanin (KLH) or bovine serum albumin (BSA).

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (See, e.g., Harlow and Lane, Antibodies: A Laboratory Manual, Cold Spring Harbor Laboratory, 1988; incorporated herein by reference).

Cytokines and Chemokines

In certain embodiments, it may be desirable to couple specific bioactive agents to one or more targeting peptides for targeted delivery to an organ or tissue. Such agents include, but are not limited to, cytokines, chemokines, pro-apoptosis factors and anti-angiogenic factors. The term “cytokine” is a generic term for proteins released by one cell population which act on another cell as intercellular mediators. Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-.alpha. and -.beta.; mullerian-inhibiting substance; mouse gonadotropin-associated peptide; inhibin; activin; vascular endothelial growth factor; integrin; thrombopoietin (TPO); nerve growth factors such as NGF-.beta.; platelet-growth factor; transforming growth factors (TGFs) such as TGF-.alpha and TGF-.beta; insulin-like growth factor-I and -II; erythropoietin (EPO); osteoinductive factors; interferons such as interferon-α, -.β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1.alpha., IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand or FLT-3, angiostatin, thrombospondin, endostatin, tumor necrosis factor and LT. As used herein, the term cytokine includes proteins from natural sources or from recombinant cell culture and biologically active equivalents of the native sequence cytokines.

Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Chemokines include, but are not limited to, RANTES, MCAF, MIP1-alpha, MIP1-Beta, and IP-10. The skilled artisan will recognize that certain cytokines are also known to have chemoattractant effects and could also be classified under the term chemokines.

Imaging Agents and Radioisotopes

In certain embodiments, the claimed peptides or proteins of the present invention may be attached to imaging agents of use for imaging and diagnosis of various diseased organs or tissues. Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

Radioisotopes of potential use as imaging or therapeutic agents include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being preferred for use in certain embodiments, and technicium^{99m} and indium¹¹¹ are also often preferred due to their low energy and suitability for long range detection.

Radioactively labeled proteins or peptides of the present invention may be produced according to well-known methods in the art. For instance, they can be iodinated by contact with sodium or potassium iodide and a chemical oxidizing agent such as sodium hypochlorite, or an enzymatic oxidizing agent, such as lactoperoxidase. Proteins or peptides according to the invention may be labeled with technetium-⁹⁹ by ligand exchange process, for example, by reducing pertechnate with stannous solution, chelating the reduced technetium onto a Sephadex column and applying the peptide to this column or by direct labeling techniques, e.g., by incubating pertechnate, a reducing agent such as SNCl₂, a buffer solution such as sodium-potassium phthalate solution, and the peptide. Intermediary functional groups which are often used to bind radioisotopes which exist as metallic ions to peptides are diethylenetriaminepentaacetic acid (DTPA) and ethylene diaminetetracetic acid (EDTA). Also contemplated for use are fluorescent labels, including rhodamine, fluorescein isothiocyanate and renographin.

In certain embodiments, the claimed proteins or peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

Cross-Linkers

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites are dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups.

Cross-linking agents may also be of use to attach chemicals, compounds, molecules or aggregates of molecules to larger particles for use in BRASIL screening.

### Magnetic Beads

It is envisioned that particles employed in the instant invention may come in a variety of sizes. While large magnetic particles (mean diameter in solution greater than 10 μm) can respond to weak magnetic fields and magnetic field gradients, they tend to settle rapidly, limiting their usefulness for reactions requiring homogeneous conditions. Large particles also have a more limited surface area per weight than smaller particles, so that less material can be coupled to them. In preferred embodiments, the magnetic beads are less than 10 μm in diameter.

Various silane couplings applicable to magnetic beads are discussed in U.S. Pat. No. 3,652,761, incorporated herein by reference. Procedures for silanization known in the art generally differ from each other in the media chosen for the polymerization of silane and its deposition on reactive surfaces. Organic solvents such as toluene (Weetall, 1976), methanol, (U.S. Pat. No. 3,933,997) and chloroform (U.S. Pat. No. 3,652,761) have been used. Silane deposition from aqueous alcohol and aqueous solutions with acid have also been used.

Ferromagnetic materials in general become permanently magnetized in response to magnetic fields. Materials termed “superparamagnetic” experience a force in a magnetic field gradient, but do not become permanently magnetized Crystals of magnetic iron oxides may be either ferromagnetic or superparamagnetic, depending on the size of the crystals. Superparamagnetic oxides of iron generally result when the crystal is less than about 300 angstroms (Å) in diameter, larger crystals generally have a ferromagnetic character.

Dispersible magnetic iron oxide particles reportedly having 300 Å diameters and surface amine groups are prepared by base precipitation of ferrous chloride and ferric chloride (Fe²⁺/Fe³⁺=1) in the presence of polyethylene imine, according to U.S. Pat. No. 4,267,234. These particles are exposed to a magnetic field three times during preparation and are described as redispersible. The magnetic particles are mixed with a glutaraldehyde suspension polymerization system to form magnetic polyglutaraldehyde microspheres with reported diameters of 0.1 μm. Polyglutaraldehyde microspheres have conjugated aldehyde groups on the surface which can form bonds to amino containing molecules such as proteins.

While a variety of particle sizes are envisioned to be applicable in the disclosed method, in a preferred embodiment, particles are between about 0.1 and about 1.5 μm diameter. Particles with mean diameters in this range can be produced with a surface area as high as about 100 to 150 m²/gm, which provides a high capacity for bioaffinity adsorbent coupling. Magnetic particles of this size range overcome the rapid settling problems of larger particles, but obviate the need for large magnets to generate the magnetic fields and magnetic field gradients required to separate smaller particles. Magnets used to effect separations of the magnetic particles of this invention need only generate magnetic fields between about 100 and about 1000 Oersteds. Such fields can be obtained with permanent magnets which are preferably smaller than the container which holds the dispersion of magnetic particles and thus, may be suitable for benchtop use. Although ferromagnetic particles may be useful in certain applications of the invention, particles with superparamagnetic behavior are usually preferred since superparamagnetic particles do not exhibit the magnetic aggregation associated with ferromagnetic particles and permit redispersion and reuse.

The method for preparing the magnetic particles may comprise precipitating metal salts in base to form fine magnetic metal oxide crystals, redispersing and washing the crystals in water and in an electrolyte. Magnetic separations may be used to collect the crystals between washes if the crystals are superparamagnetic. The crystals may then be coated with a material capable of adsorptively or covalently bonding to the metal oxide and bearing functional groups for coupling with various target molecules.

### Non-Magnetic Beads, Flow Cytometry and FACS

In another embodiment, the target of interest may be non-covalently or covalently attached to non-magnetic beads, such as glass, polyacrylamide, polystyrene or latex. Targets may be attached to such beads by the same techniques discussed above for magnetic beads. After exposure of bead to phage library, those phage bound to beads may be separated from unbound phage by, for example, centrifugation.

In certain embodiments, cells to be screened by BRASIL may be presorted using some form of flow cytometry. Non-limiting examples of flow cytometry methods are disclosed in Betz et al. (1984), Wilson et al. (1988), Scillian et al. (1989), Frengen et al. (1994), Griffith et al. (1996), Stuart et al. (1998) and U.S. Pat. Nos. 5,853,984 and 5,948,627, each incorporated herein by reference in its entirety. U.S. Pat. Nos. 4,727,020, 4,704,891 and 4,599,307, incorporated herein by reference, describe the arrangement of the components comprising a flow cytometer and the general principles of its use.

In the flow cytometer, beads, cells or other particles are passed substantially one at a time through a detector, where each particle is exposed to an energy source. The energy source generally provides excitatory light of a single wavelength. The detector comprises a light collection unit, such as photomultiplier tubes or a charge coupled device, which may be attached to a data analyzer such as a computer. The beads, cells or particles can be characterized by their response to excitatory light, for example by detecting and/or quantifying the amount of fluorescent light emitted in response to the excitatory light. Beads or cells exhibiting a particular characteristic can be sorted using an attached cell sorter, such as the FACS Vantage™ cell sorter sold by Becton Dickinson Immunocytometry Systems (San Jose, Calif.).

### Nucleic Acids

Nucleic acids according to the present invention may encode a targeting peptide, a receptor protein or a fusion protein. The nucleic acid may be derived from genomic DNA, complementary DNA (cDNA) or synthetic DNA. Where incorporation into an expression vector is desired, the nucleic acid may also comprise a natural intron or an intron derived from another gene. Such engineered molecules are sometime referred to as “mini-genes.”

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater nucleotide residues in length.

It is contemplated that targeting peptides, fusion proteins and receptors may be encoded by any nucleic acid sequence that encodes the appropriate amino acid sequence. The design and production of nucleic acids encoding a desired amino acid sequence is well known to those of skill in the art, using standardized codon tables (see Table 2 below). In preferred embodiments, the codons selected for encoding each amino acid may be modified to optimize expression of the nucleic acid in the host cell of interest. Codon preferences for various species of host cell are well known in the art.

TABLE 2

| | |
|---|---|
| Amino Acid | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACG ACG ACU |
| Valine | Val | V | GUA GUC GUG GUU |
| Tryptophan | Trp | W | UGG |
| Tyrosine | Tyr | Y | UAC UAU |

In addition to nucleic acids encoding the desired targeting peptide, fusion protein or receptor amino acid sequence, the present invention encompasses complementary nucleic acids that hybridize under high stringency conditions with such coding nucleic acid sequences. High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture.

### Vectors for Cloning, Gene Transfer and Expression

In certain embodiments expression vectors are employed to express the targeting peptide or fusion protein, which can then be purified and used. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are known.

Regulatory Elements

The terms “expression construct” or “expression vector” are meant to include any type of genetic construct containing a nucleic acid coding for a gene product in which part or all of the nucleic acid coding sequence is capable of being transcribed. In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, rat insulin promoter, and glyceraldehyde-3-phosphate dehydrogenase promoter can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

Where a cDNA insert is employed, typically one will typically include a polyadenylation signal to effect proper polyadenylation of the gene transcript. The nature of the polyadenylation signal is not believed to be crucial to the successful practice of the invention, and any such sequence may be employed, such as human growth hormone and SV40 polyadenylation signals. Also contemplated as an element of the expression construct is a terminator. These elements can serve to enhance message levels and to minimize read through from the construct into other sequences.

Selectable Markers

In certain embodiments of the invention, the cells containing nucleic acid constructs of the present invention may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin, and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome, and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Preferred gene therapy vectors are generally viral vectors.

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression making them ideally suited for rapid, efficient, heterologous gene expression. techniques for preparing replication infective viruses are well known in the art.

Of course in using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A non-limiting method of purifying the vector involves the use of buoyant density gradients, such as cesium chloride gradient centrifugation.

DNA viruses used as gene vectors include the papovaviruses (e.g., simian virus 40, bovine papilloma virus, and polyoma) (Ridgeway, 1988; Baichwal and Sugden, 1986) and adenoviruses (Ridgeway, 1988; Baichwal and Sugden, 1986).

One of the preferred methods for in vivo delivery involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to express an antisense polynucleotide that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retroviral infection, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 50□-tripartite leader (TPL) sequence which makes them preferred mRNAs for translation.

In currently used systems, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of adenovirus vectors which are replication deficient depend on a unique helper cell line, designated 293, which is transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3, or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells, may be derived from the cells of other mammalian species that are permissive for human adenovirus. Such cells include, e.g., Vero cells or other monkey embryonic mesenchymal or epithelial cells. As discussed, the preferred helper cell line is 293.

Racher et al., (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) are employed as follows. A cell innoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking is initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking is commenced for another 72 hr.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

A typical vector applicable to practicing the present invention is replication defective and will not have an adenovirus E1 region. Thus, it are most convenient to introduce the polynucleotide encoding the gene at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors as described by Karlsson et al., (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Other gene transfer vectors may be constructed from retroviruses. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env. that code for capsid proteins, polymerase enzyme, and envelope components, respectively. A sequence found upstream from the gag gene contains a signal for packaging of the genome into virions. Two long terminal repeat (LTR) sequences are present at the 5□ and 3□ ends of the viral genome. These contain strong promoter and enhancer sequences, and also are required for integration in the host cell genome (Coffin, 1990).

In order to construct a retroviral vector, a nucleic acid encoding protein of interest is inserted into the viral genome in the place of certain viral sequences to produce a virus that is replication-defective. In order to produce virions, a packaging cell line containing the gag, pol, and env genes, but without the LTR and packaging components, is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are capable of infecting a broad variety of cell types. However, integration and stable expression require the division of host cells (Paskind et al., 1975).

There are certain limitations to the use of retrovirus vectors. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This may result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs. Vectors derived from viruses such as vaccinia virus (Ridgeway, 1988; Baichwal and Sugden, 1986; Coupar et al., 1988), adeno-associated virus (AAV) (Ridgeway, 1988; Baichwal and Sugden, 1986; Hermonat and Muzycska, 1984), and herpes viruses may be employed. They offer several attractive features for various mammalian cells (Friedmann, 1989; Ridgeway, 1988; Baichwal and Sugden, 1986; Coupar et al., 1988; Horwich et al., 1990).

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990), DEAE-dextran (Gopal, 1985), electroporation (Tur-Kaspa et al., 1986; Potter et al., 1984), direct microinjection, DNA-loaded liposomes and lipofectamine-DNA complexes, cell sonication, gene bombardment using high velocity microprojectiles, and receptor-mediated transfection (Wu and Wu, 1987; Wu and Wu, 1988). Some of these techniques may be successfully adapted for in vivo or ex vivo use.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa, and hepatoma cells. Nicolau et al., (1987) accomplished successful liposome-mediated gene transfer in rats after intravenous injection.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr: that confers resistance to methotrexate; gpt, that confers resistance to mycophenolic acid; neo, that confers resistance to the aminoglycoside G418; and hygro, that confers resistance to hygromycin.

### Pharmaceutical Compositions

Where clinical applications are contemplated, it is necessary to prepare pharmaceutical compositions—expression vectors, virus stocks, proteins, antibodies and drugs—in a form appropriate for the intended application. Generally, this will entail preparing compositions that are essentially free of impurities that could be harmful to humans or animals.

One generally will desire to employ appropriate salts and buffers to render delivery vectors stable and allow for uptake by target cells. Buffers also are employed when recombinant cells are introduced into a patient. Aqueous compositions of the present invention comprise an effective amount of the protein or peptide, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the proteins or peptides of the present invention, its use in therapeutic compositions is contemplated. Supplementary active ingredients also can be incorporated into the compositions.

The active compositions of the present invention may include classic pharmaceutical preparations. Administration of these compositions according to the present invention are via any common route so long as the target tissue is available via that route. This includes oral, nasal, buccal, rectal, vaginal or topical. Alternatively, administration may be by orthotopic, intradermal, subcutaneous, intramuscular, intraperitoneal, intraarterial or intravenous injection. Such compositions normally would be administered as pharmaceutically acceptable compositions, described supra.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it are preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

### Therapeutic Agents

In certain embodiments, chemotherapeutic agents may be attached to a targeting peptide or fusion protein for selective delivery to a tumor. Agents or factors suitable for use include any chemical compound that induces DNA damage when applied to a cell. Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. An alkylating agent, may include, but is not limited to, a nitrogen mustard, an ethylenimene, a methylmelamine, an alkyl sulfonate, a nitrosourea or a triazines. They include but are not limited to: busulfan, chlorambucil, cisplatin, cyclophosphamide (cytoxan), dacarbazine, ifosfamide, mechlorethamine (mustargen), and melphalan.

Antimetabolites

Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

Natural Products

Natural products generally refer to compounds originally isolated from a natural source, and identified has having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. They include such compounds as vinblastine (VLB) and vincristine.

Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Examples of antitumor antibiotics include, but are not limited to, bleomycin, dactinomycin, daunorubicin, doxorubicin (Adriamycin), plicamycin (mithramycin) and idarubicin.

Hormones

Corticosteroid hormones are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

Progestins such as hydroxyprogesterone caproate, medroxyprogesterone acetate, and megestrol acetate have been used in cancers of the endometrium and breast. Estrogens such as diethylstilbestrol and ethinyl estradiol have been used in cancers such as breast and prostate. Antiestrogens such as tamoxifen have been used in cancers such as breast. Androgens such as testosterone propionate and fluoxymesterone have also been used in treating breast cancer. Antiandrogens such as flutamide have been used in the treatment of prostate cancer. Gonadotropin-releasing hormone analogs such as leuprolide have been used in treating prostate cancer.

Miscellaneous Agents

Some chemotherapy agents do not qualify into the previous categories based on their activities. They include, but are not limited to, platinum coordination complexes, anthracenedione, substituted urea, methyl hydrazine derivative, adrenalcortical suppressant, amsacrine, L-asparaginase, and tretinoin. It is contemplated that they are included within the compositions and methods of the present invention.

Platinum coordination complexes include such compounds as carboplatin and cisplatin (cis-DDP).

An anthracenedione such as mitoxantrone has been used for treating acute granulocytic leukemia and breast cancer. A substituted urea such as hydroxyurea has been used in treating chronic granulocytic leukemia, polycythemia vera, essental thrombocytosis and malignant melanoma. A methyl hydrazine derivative such as procarbazine (N-methylhydrazine, MIH) has been used in the treatment of Hodgkin's disease. An adrenocortical suppressant such as mitotane has been used to treat adrenal cortex cancer, while aminoglutethimide has been used to treat Hodgkin's disease.

Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

Non-limiting examples of pro-apoptosis agents contemplated within the scope of the present invention include gramicidin, magainin, mellitin, defensin, cecropin, (KLAKLAK)₂ (SEQ ID NO:1), (KLAKKKLA)₂ (SEQ ID NO:2), (KAAKKAA)₂ (SEQ ID NO:3) or (KLGKKLG)₃ (SEQ ID NO:4).

Angiogenic Inhibitors

In certain embodiments the present invention may concern administration of targeting peptides attached to anti-angiogenic agents, such as angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline.

### Dosages

The skilled artisan is directed to “Remington's Pharmaceutical Sciences” 15th Edition, chapter 33, and in particular to pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA Office of Biologics standards.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### BRASIL

Probing molecular diversity at the cell surface level is important for the identification of targeting peptides and the development of targeted therapies. As opposed to purified receptors, membrane-bound proteins are more likely to preserve their functional conformation. Many cell surface receptors require homo- or hetero-dimeric interactions that occur only within the cell membrane environment. Combinatorial approaches allow the selection of cell membrane ligands in an unbiased functional assay, without any preconceived notions about the nature of the cellular receptors. Thus, unknown receptors can be targeted. Despite these advantages, it is often difficult to isolate specific ligands due to the high complexity of targets expressed simultaneously on a given cell population.

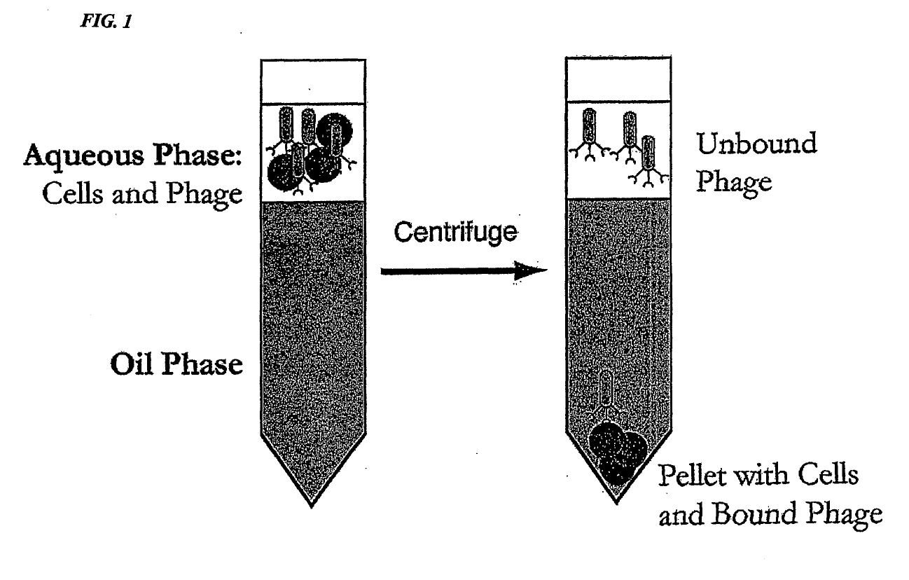

To address these problems, a new in vitro approach has been developed to improve the selection of phage at the level of single cells or small clumps of cells. This method, Biopanning and Rapid Analysis of Selective Interactive Ligands (BRASIL), is based on a procedure that allows cell-phage complexes to be separated from the remaining unbound phage in a single differential centrifugation step (FIG. 1). As described herein, BRASIL has been successfully used to isolate phage in various cell systems.

BRASIL involves the addition of cells to centrifuge tubes containing an first (preferably aqueous) phase layered over a second (preferably organic) phase, as described below. Upon centrifugation, the cells and any bound phage end up in a pellet at the bottom of the second phase, while non-bound phage remain in the upper first phase. This gentle separation technique helps to preserve the phage-receptor interaction for targeting peptides that are not tightly bound to receptor.

In preferred embodiments, the first phase is aqueous and the second phase is organic. As organic phases are generally immiscible with aqueous phases, this prevents mixing and dilution of the phase components and consequent changes in density. Use of an aqueous phase for binding of phage to cells is preferred, as it mimics the in vivo environment in which protein interactions normally occur. An organic second phase is also preferred since it is likely to reduce background by interfering with non-specific hydrophobic interactions, while retaining specifically bound phage by increasing the strength of ionic interactions or hydrogen bonding.

BRASIL may be used to isolate phage displaying peptide sequences that bind to specific markers of different cell subpopulations from any selected organ, tissue or cell type. Cell subpopulations may be purified ex-vivo by Ficoll gradient and/or identified by Fluorescent Activated Cell Analysis SACS) before the BRASIL method is implemented. This improved in vitro panning method may be used to retrieve phage that bind to markers found only in certain cell subpopulations. Fine Needle Aspirations (FNAs) of organs are excellent sources of cells to perform biopanning with BRASIL. The skilled artisan will realize that the BRASIL technique is applicable for identifying targeting peptides directed against a wide range of organs, tissues and cell types.

### Materials and Methods

Reagents and Cells

A phage library displaying random cyclic peptides with the structure CX₆C(C, cysteine; X, any amino acid residue) was used for the screenings. Phage libraries and clones were produced according to Koivunen et al. (1999), using known methods (Smith, 1985; Smith and Scott 1993). Kaposi's sarcoma cells (KS1767 cell line) were maintained in minimal essential medium (MEM) supplemented with 10% fetal calf serum (Gibco-BRL, Rockville, Md.). Dibutyl phthalate, and cyclohexane (Sigma-Aldrich, St. Louis, Mo.) were obtained commercially. Peptides used were synthesized to greater than 95% purity, cyclized, and analyzed by HPLC and mass spectrometry (AnaSpec, San Jose, Calif.).

Ex-Vivo Procedure

In an exemplary in vitro procedure, prostate cells were harvested, washed and re-suspended in medium containing 1% BSA (100-300 μl). A phage library (10⁹ phage) was added and left on ice for 4 h. After transfer to a 400 μl eppendorf tube containing 200 μl of dibutylphtalate (Sigma), the mixture was centrifuged for 10 min at 10,000 g. The tube bottom (containing the cell-phage complexes) was snap-frozen in liquid nitrogen to prevent cross-contamination with unbound phage in the upper aqueous phase. The frozen tube was carefully cut with a sharp razor blade and the pellet was transferred to a fresh Falcon tube. PBS (100-200 μl) was added and the pellet homogenized by repeated pipeting. After adding 1 ml of E. coli K91kan bacteria and incubation for 1 hr, LB containing tetracycline was added and the admixture was grown overnight in a 37° C. shaker. Phage bound to pelleted cells were recovered as bacterial plaques. Careful steps were taken to prevent any aggregates that could bias the biopanning results. The BSA-containing media was filtered through a 0.22 micron sterile mesh and the phage centrifuged in solution for 5 min at 16,000 g immediately prior to addition to the cells. This study showed that bound phage could be harvested from prostate cells using the BRASIL method (not shown).

BRASIL Method Optimization

Cells were harvested with phosphate-buffered saline (PBS) and 5 mM EDTA, washed with MEM, and re-suspended in MEM containing 1% BSA at 10⁶ cells/ml and incubated with phage in 1.5 ml Eppendorf tubes. After 4 h, 100 μl of the cell-phage suspension was gently transferred to the top of a non-miscible organic lower phase (200 μl in a 400 μl-Eppendorf tube) and centrifuged at 10,000 g for 10 minutes. The preferred organic phase combination consisted of a mixture of dibutyl phthalate:cyclohexane (9:1 [v/v]; ρ=1.03 g/ml). BRASIL has been attempted with other phthalate admixtures with the appropriate density (for example, dibutyl phthalate:diisooctyl phthalate; 4:6 [v/v]) with similar results. The tube was snap frozen in liquid nitrogen, the bottom of the tube sliced off, and the cell-phage pellet transferred to a new tube. Bound phage were rescued by infection with 200 μl of E. coli K91kan host bacteria in log phase. To evaluate binding specificity, phage and cells were incubated with the cognate or control synthetic peptides for competition assays.

Binding Assays with Phage Clones

KS1767 cells were detached with cold EDTA and re-suspended in MEM containing 1% BSA. RGD-4C phage (Pasqualini et al., 1997) were used as a defined ligand that displays a specific αv integrin-binding motif. The cell suspension was incubated with RGD-4C phage or a control phage with no peptide insert (fd-tet phage). Increasing amounts of either phage were added to the cells in suspension and the cell-phage admixture was incubated for 4 hr on ice. BRASIL was performed on ice to minimize post-binding events such as ligand-receptor internalization by the target cells. The cells were separated by centrifugation through the organic phase as described above. Bound phage were recovered and phage TU were counted. To compare BRASIL to conventional cell panning methods that require an additional washing step, 200 μl of the cell suspension were incubated with phage for 4 hr on ice. Unbound phage from 100 μl aliquots were removed either by centrifuging over the organic phase or by washing the cells three times with 1 ml of PBS containing 0.3% BSA. Each condition was repeated at least three times. Competitive inhibition was tested with the synthetic RGD-4C peptide, containing the targeting sequence CDCRGDCFC (SEQ ID NO:9) were compared to control peptides CARAC (SEQ ID NO:5) or GRGESP (SEQ ID NO:10) used at the same molar ratios.

### Results

The BRASIL technique was tested using RGD4C phage that bind to alpha-v integrins (Pasqualini et al., 1997) and the KS1767 cell line, which expresses high levels of alpha-v integrins. It was first determined if the oil mixture would interfere with the infection rate. Increasing amounts of oil were added to a bacterial culture and phage added to them. After 1 hr infection, the cells were plated and the number of tetracycline resistant colonies (infected by phage) counted. No significant difference between the control (no oil added) and the oil mixtures could be detected (data not shown) suggesting that the oil mixture does not interfere with the infection rate and recovery of phage.

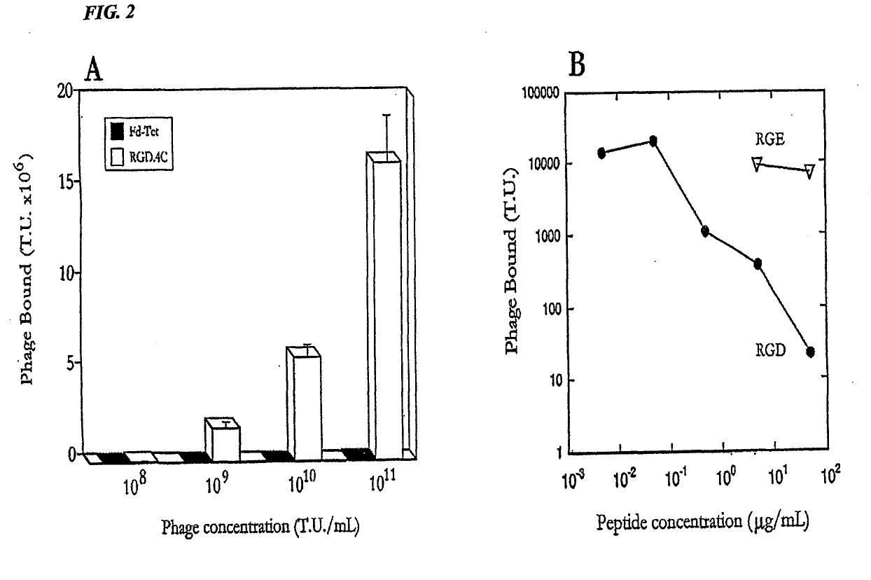

It was determined whether phage would pellet at the bottom of the tube if no cells were present. 10⁹ TU of Fd (insertless) phage were added to the medium and centrifuged. No Fd phage could be detected at the bottom of the tube or in the oil phase. Next, it was tested if phage could be carried specifically by the cells to the oil phase and then recovered by infection with bacteria. For this, increasing amounts of RGD4C phage or Fd phage were added to KS1767 cells in suspension, incubated for 4 hr on ice and then centrifuged over the oil. As shown in FIG. 2A, the number of phage recovered from the cells increased with the number of phage added. The ratio between the number of RGD4C to Fd phage recovered (enrichment) varied consistently from 100 to 500-fold. The binding of the RGD4C phage to the KS1767 cells was specific and was mediated by the peptide expressed in the pIII protein, since a competition experiment with the corresponding soluble peptide completely inhibited the binding of the RGD4C phage binding to KS1767 cells, bringing the number of phage bound close to the number of Fd phage (background) (FIG. 2B). Negative control peptides (CARAC, SEQ ID NO:5 or GRGESP, SEQ ID NO:10) had no effect on RGD4C phage binding to KS1767 cells (not shown). The recovery of phage with or without the snap-freeze step was compared. No substantial decrease was noted in the amounts of test phage recovered (data not shown).

The recovery of phage with BRASIL was compared to standard biopanning methods requiring a washing step. The number of RGD-4C phage recovered by BRASIL was significantly higher (t test, P<0.01) than the number of the same phage recovered when a conventional phage-cell binding strategy involving washing was used (not shown). Conversely, significantly lower background (t test, P<0.01) with the negative control phage was observed (not shown). Given the significant increase in recovery of specific phage and the substantial decrease in background, the overall accuracy improved consistently by more than one order of magnitude when BRASIL was used relative to conventional cell panning methods.

### Example 2

### VEGF Targeting Peptide Identified by BRASIL

Diabetic retinopathy is the formation of new blood vessels (angiogenesis) in the retina and cornea, induced by hyperglycemia. Although the neovascularization process of the retina is not fully understood, growth factors, especially vascular endothelial growth factors (VEGFs) play an important role in this process.

Intraocular neovascularization is a pathological complication of many eye diseases and is the leading cause of blindness in the world. Although hyperglycemia per se seems to be the main cause of diabetic retinopathy (Engerman and Kern, 1986), it is the induction of growth factors that start the angiogenic process. Among several possible candidates, vascular endothelial growth factor (VEGF) seems to be most important mediator of the ischemia-induce neovascularization since several anti-VEGF therapies prevent ocular angiogenesis in animal models. VEGF is produced by several retinal cell types (ganglion cells, RPE, pericytes, endothelial cells, astrocytes and Müller cells). Its expression is upregulated by hypoxia and it diffuses freely through the eye.

Angiogenesis is a complex process, which seems to be balanced by the presence of activators and inhibitors (Hanahan and Folkman, 1996). VEGF is a major activator and regulator of both physiological and pathological neovascularization (reviewed by Ferrara and Davis-Smyth, 1997). It is a relative specific mitogen for vascular endothelial cells and elicits a pronounced angiogenic response in a variety of in vivo models. VEGF belongs to a multigene family with 5 members described so far: VEGF, VEGF-B, VEGF-C, VEGF-D, PIGF and the orf virus VEGF (also called VEGF-E). Alternative exon splicing of the genes produces multiple species of mRNA with distinct biological effects (Tischer et al., 1991; Veikkcola and Alitalo, 1999). VEGFs are also produced as homo- and heterodimers, although very little is known about the function of the VEGF heterodimers.

### Materials and Methods

Cells and Reagents

Recombinant human VEGF₁₆₅ (Pharmingen, San Diego, Calif.), recombinant human VEGFR-1 (Oncogene Research Products, Boston, Mass.), recombinant rat NRP-1/Fc, rat NRP-2/Fc, human VEGFR-2/Fc (all three receptor/chimeras with the Fc region of human IgG₁), PDGF-BB, anti-VEGFR-1 (polyclonal anti-Flt1), and anti-human VEGF polyclonal antibody (R&D Systems Minneapolis, Minn.) were all obtained commercially. HUVEC (human umbilical vein endothelial cells) were purchased from Clonetics and cultured according to the manufacturer's instructions. Anti-mouse CD13 antibodies R3-63 and 2M-7 were produced and characterized as described (Hansen et al., 1993). The anti-M13 polyclonal antibody (Amersham-Pharmacia) was obtained commercially. Anti-CD31 antibody was purchased from Pharmingen (CA), anti-smooth muscle actin conjugated to Cy3 or FITC was purchased from Sigma. Anti-desmin polyclonal serum was purchased from Daiko. Aminopeptidase-N (leucine aminopeptidase) was purchased from Sigma. HUVEC were cultured and used between passages 2 and 8, according to the manufacturer's protocol (Clonetics, San Diego, Calif.). In order to minimize receptor-mediated internalization, cells and media were kept on ice unless otherwise stated.

BRASIL

Cells were harvested with PBS, 5 mM EDTA (5 minutes), washed with PBS and ressuspended in MEM containing 1% BSA (MEM 1% BSA) at 10⁶ cells/ml. Phage was added to the cell suspension and incubated on ice. After 4 hr, 100 μl of the cell suspension was transferred to 400 μl eppendorf tubes containing 200 μl of dibutyl phthalate:cyclohexane mixture (9:1) and centrifuged at 10.000 g for 10 minutes. Cells with bound phage migrated to the bottom of the tube within the oil phase and the unbound phage remained at the top of the oil in the soluble phase. The tubes were snap-frozen in liquid N₂, the pellet cut off, transferred to a new eppendorf and phage rescued by infection with 200 μL of E. coli K91kan cells in log-phase, then diluted and plated onto LB plates supplemented with tetracycline.

HUVEC Biopanning by BRASIL Phage Display

A two-step biopanning strategy was designed to isolate phage that bind to angiogenic endothelial cells. To decrease non-specific binding, the phage library was pre-cleared on starved HUVEC cells before panning on the same cell line stimulated with VEGF₁₆₅. After centrifugation through the organic phase, phage bound to the VEGF₁₆₅-stimulated HUVEC pellet were recovered by bacterial infection, amplified, and subjected to two more rounds of selection.

Phage peptide libraries were obtained, expanded and manipulated as described (Pasqualini et al., 1999). HUVEC at 80% confluence cultured in endothelial basal medium (EBM-2; Clonetics) without supplements for 24 hr were defined as “starved HUVEC.” The medium was then replaced by EBM-2 supplemented with 20 ng/ml VEGF₁₆₅ and the cells cultured under these conditions for another 18 hr were defined as “VEGF₁₆₅-stimulated HUVEC.” Both, starved and VEGF₁₆₅-stimulated HUVEC were harvested with ice-cold PBS and 5 mM EDTA, washed once with EBM-2 plus 1% BSA, and re-suspended in the same medium at 10⁷ cells/ml. In the pre-clearing step, starved HUVEC (10⁶ cells) were incubated with 10⁹ TU of unselected CX₆C phage library for 2 hr on ice; the mixture was then centrifuged through the organic phase. In a screening step, the unbound phage left over in the aqueous upper phase (supernatant) was transferred to a fresh tube and incubated with VEGF₁₆₅-stimulated HUVEC (10⁶ cells). After 4 hr on ice, the cell-phage complexes were separated by centrifugation through the organic lower phase. The phage population in the cell pellet was recovered by infection of 200 μl of E. coli K91kan host bacteria growing in log phase. This procedure was repeated 3 times using the phage obtained from the previous round. After the third round of biopanning, 32 phage were randomly selected and sequenced for analysis.

Binding Assays on Purified Receptors.

Human VEGFR-1, human VEGFR-2, rat NRP-1, and rat NRP-2 (1 μg in 50 μl of PBS) were immobilized on microtiter well plates overnight at 4° C. The wells were washed twice with PBS, blocked with PBS containing 3% BSA for 2 h at room temperature, and then incubated with 10⁹ TU of either CPQPRPLC (SEQ ID NO:6) phage, CNIRRQGC (SEQ ID NO:11) phage, or fd-tet phage in 50 μl of PBS/1.5% BSA. After 1 hr at room temperature, wells were washed nine times with PBS and phage were recovered by bacterial infection. Serial dilutions were plated onto Luria-Bertani (LB) medium supplemented with tetracycline. VEGF₁₆₅, PDGF-BB, or synthetic peptides were used at the indicated concentrations and pre-incubated with the immobilized proteins to evaluate competitive inhibition of phage binding. ELISA with either polyclonal anti-VEGFR-1 serum or anti-human IgG (VEGFR-2, NRP-1, and NRP-2) confirmed the presence and concentration of the immobilized receptors on the microtiter plates. To show that the VEGF receptors were functionally active, VEGF₁₆₅ (50 ng/ml) was incubated with the immobilized receptors for 2 hr at room temperature. Following three washes, VEGF₁₆₅ binding was evaluated by ELISA by using anti-VEGF specific antibodies (data not shown).

### Results

Biopanning on VEGF Stimulated HUVEC

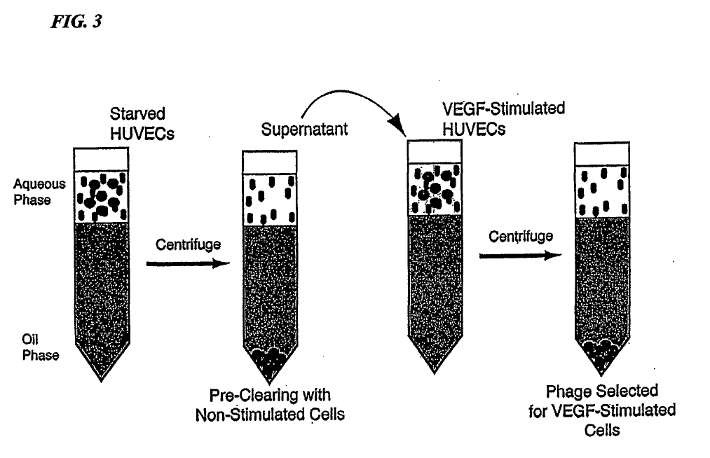

An advantage of BRASIL is that the unbound phage left in the upper aqueous phase can be used for a new round of panning with minimal loss. This approach was used to first pre-clear the phage display library with starved HUVEC before biopanning with VEGF₁₆₅-activated HUVEC (FIG. 3). The VEGF₁₆₅-activated cells were then collected by BRASIL and the phage bound to them amplified and submitted to another round of selection.

To test the selection method, 21 phage randomly chosen clones were examined for binding to starved HUVEC and to VEGF-stimulated HUVEC. Fourteen out of 21 clones (67%) had a greater than 150% enhancement (range, 1.5 to 8.7-fold; median, 2.2-fold) in the ratio of cell binding upon VEGF stimulation normalized to control insertless phage (data not shown). After three rounds of BRASIL selection on VEGF₁₆₅-activated cells, 34 phage were randomly selected for sequencing. Alignment analysis of the 34 insert sequences revealed that 24 clones (70%) of the phage recovered by BRASIL displayed peptide motifs that could be mapped to sequences present in VEGF family members (not shown). Peptides with homology to the VEGF family are shown in Table 3 below.

A phage clone displaying a peptide sequence CPQPRPLC (SEQ ID NO:6, referred to hereafter as “clone 19”) was very similar in sequence to a portion of the VEGF-B isoform 167 protein. Three different peptides contained the motif IRR^{E}/_{Q}. The motif IRR^{E}/_{Q} did not show substantial homology with known protein sequences and further experiments focussed on CPQPRPLC (SEQ ID NO:6).

TABLE 3

Targeting peptides with
homology to VEGF family members

| | | |
|---|---|---|
| Clone # | Sequence | Homologies |

| | | | |
|---|---|---|---|
| Peptide #1 | CEGESASC | SEQ ID NO:40 | VEGF-D |
| Peptide #3 | CVPMRLQC | SEQ ID NO:41 | VEGF-A, VEGF-B, |
| | | | VEGF-C, PIGF-1, |
| | | | PIGF-2 |
| Peptide #4 | CLGKGSVC | SEQ ID NO:42 | VEGF-A, VEGF-D |
| Peptide #6 | CLSPIGEC | SEQ ID NO:43 | VEGF-A |
| Peptide #7 | CNLSVPAC | SEQ ID NO:44 | VEGF-A, VEGF-D |
| Peptide #9 | CIIGSYVC | SEQ ID NO:45 | PIGF-1, PIGF-2 |
| Peptide #11 | CADVLRPC | SEQ ID NO:46 | VEGF-D |
| Peptide #12 | CWRSVEVC | SEQ ID NO:47 | VEGF-B, VEGF-C |
| Peptide #13 | CSIRRESC | SEQ ID NO:48 | VEGF-C, VEGF-D |
| Peptide #17 | CAVVFSQC | SEQ ID NO:49 | VEGF-B |
| Peptide #18 | CLANLQTC | SEQ ID NO:50 | VEGF-A, VEGF-C |
| Peptide #19 | CPQPRPLC | SEQ ID NO:6 | VEGF-B, PIGF-1, |
| | | | PIGF-2 |
| Peptide #21 | CNIRRQGC | SEQ ID NO:51 | VEGF-C, VEGF-D |
| Peptide #23 | CIRREKRC | SEQ ID NO:52 | VEGF-C, VEGF-D, |
| | | | PIGF-1, PIGF-2 |
| Peptide #24 | CAGKSSNC | SEQ ID NO:53 | VEGF-D |
| Peptide #25 | CRECGERC | SEQ ID NO:54 | PIGF-1 |
| Peptide #26 | CMARQARC | SEQ ID NO:55 | VEGF-A |
| Peptide #28 | CLPISSSC | SEQ ID NO:56 | VEGF-D |
| Peptide #29 | CGRAKVRC | SEQ ID NO:57 | PIGF-1, PIGF-2 |
| Peptide #30 | CASGSENC | SEQ ID NO:58 | VEGF-D |
| Peptide #31 | CMRGKGLC | SEQ ID NO:59 | VEGF-A, PIGF-2 |
| Peptide #32 | CAGGGAYC | SEQ ID NO:60 | VEGF-A, VEGF-B |
| Peptide #33 | CAAAPIRC | SEQ ID NO:61 | VEGF-B, VEGF-C |
| Peptide #36 | CGRDSKQC | SEQ ID NO:62 | VEGF-D |

Other HUVEC binding peptides that were not homologous to VEGF included CVFAILAC (SEQ ID NO:128), CGVQYVNC (SEQ ID NO:129), CSYKANSC (SEQ ID NO:130), CYQSSSGC (SEQ ID NO:131), CRGGGRLC (SEQ ID NO:132), CGSDRWLC (SEQ ID NO:133), CLVYNPAC (SEQ ID NO:134), CIPGTSLC (SEQ ID NO:135), CATEAVGC (SEQ ID NO:136) and CWGGNQAC (SEQ ID NO:137).

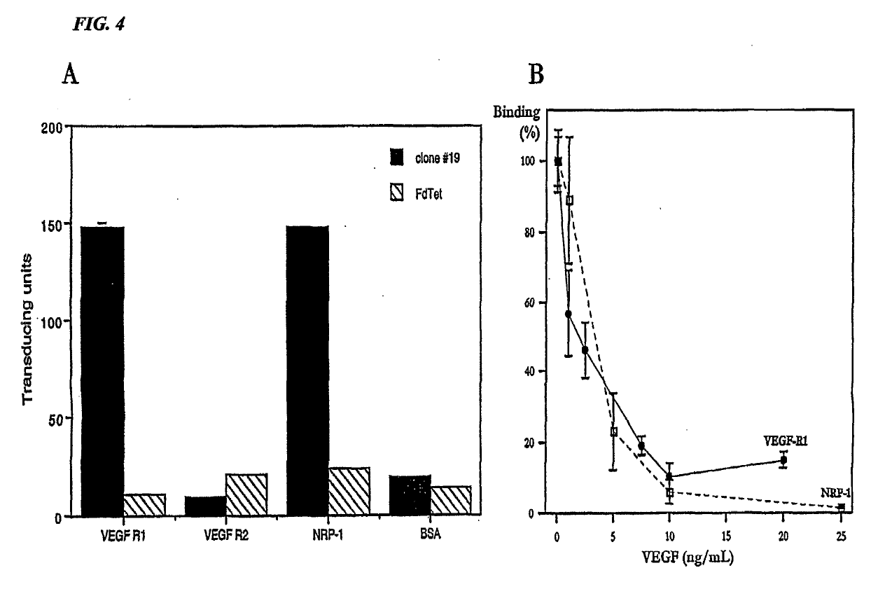

In vitro phage display was used with different recombinant VEGF receptors to determine if the clone 19 peptide bound to one or more of the VEGF receptors. As shown in FIG. 4, clone-19 bound to human VEGF-R1 as well as to rat Neuropilin-1 (NRP-1) but not to the human VEGF-R2. This result is consistent with the binding profile of VEGF-B (Olofsson et al., 1999). The lack of binding to VEGF-R2 was not due to absence of activity, since all three immobilized receptors showed similar VEGF₁₆₅ binding activity (data not shown). The clone 19 phage exhibited over a 1,000-fold enrichment of binding to VEGF-R1 over fd-tet phage (not shown). Clone 19 phage did not bind to the neutropilin-2 (NRP-2) receptor (not shown).

The VEGF₁₆₅ and VEGF-B isoforms are known to compete for binding to VEGF-R1 (Olofsson et al., 1999). The interaction of clone-19 with VEGF-R1 and NRP-1 could be blocked by competition with VEGF₁₆₅ (FIG. 4A) but not by up to 200 ng/ml of PDGF-BB (data not shown). The competition with VEGF₁₆₅ was concentration dependent and 100% inhibition was obtained with as low as 10 ng/ml of VEGF₁₆₅ (FIG. 4B). Binding of clone 19 phage could also be blocked by the cognate peptide CPQPRPLC (SEQ ID NO:6), but with differential effects (not shown). The CPQPRPLC (SEQ ID NO:6) peptide was approximately 100-fold more efficient in blocking phage binding to VEGF-R1 than to NRP-1 (not shown).

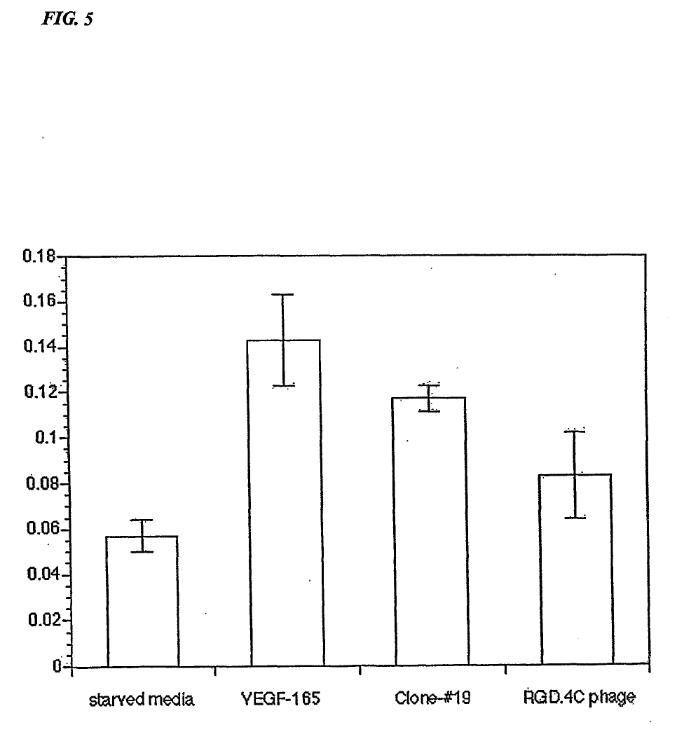

These results show that CPQPRPLC (SEQ ID NO:6) is a chimeric VEGF-B-family mimeotope that interacts specifically with VEGFR-1 and NRP-1. VEGF-B₁₆₇ is a possible mitogen for HUVEC cells (Olofsson et al, 1996). As shown in FIG. 5, 10¹⁰ T.U. of phage clone-19 significantly induced proliferation of HUVEC compared to unstimulated cells or the RGD4C phage, which also binds to HUVEC.

VEGF-B has two mRNA splice variants generated by the use of different, but overlapping, reading frames of exon 6 (isoforms 167 and 186), which diverge in sequence in their carboxy termini (Olofsson et al., 1999). The pentapeptide sequence PRPLC is found in the VEGF-B₁₆₇ carboxy terminus region encoded by exon 6B, starting at the second residue after the boundary between exons 5 and 6B. PRPLC is a neuropilin-1 (NRP-1) binding domain (Makinen et al, 1999). On the other hand, the tetrapeptide sequence PQPR, which overlaps with PRPLC and also with the clone 19 peptide, is found in the carboxy terminal of VEGF-B₁₈₆, and is encoded by exon 6A. PQPR is embedded within a 12-residue known NRP-1 binding site (Makinen et al, 1999).

HUVEC cells were also panned against a CX7C phage library. The targeting phage peptide sequences identified are shown in Table 4 below.

TABLE 4
CX7C Peptides binding to HUVEC cells.

| | |
|---|---|
| CISWWFWSC | SEQ ID NO:138 |
| CEWSGIWAC | SEQ ID NO:139 |
| CNPLFWWWC | SEQ ID NO:140 |
| CGGWLFPPC | SEQ ID NO:141 |
| CEWWPEWLC | SEQ ID NO:142 |
| CARYLWSWC | SEQ ID NO:143 |
| CAWWRFGLC | SEQ ID NO:144 |
| CRGEWGMMC | SEQ ID NO:145 |
| CFWPFESWC | SEQ ID NO:146 |
| CSNAWVHAC | SEQ ID NO:147 |
| CSWYWWLGC | SEQ ID NO:148 |
| CGGWLFPPC | SEQ ID NO:149 |
| CIEWGSRDC | SEQ ID NO:150 |
| CVRSSVVAC | SEQ ID NO:151 |
| CEDSSRANC | SEQ ID NO:152 |
| CGGWLFPPC | SEQ ID NO:153 |
| CLLVGQVRC | SEQ ID NO:154 |
| CPRYLFWLC | SEQ ID NO:155 |
| CYRSAGAGC | SEQ ID NO:156 |
| CGGWLFPPC | SEQ ID NO:157 |
| CTRVGPKRC | SEQ ID NO:158 |
| CKSGQIAVC | SEQ ID NO:159 |
| CWWPWGGWC | SEQ ID NO:160 |
| CDWGLWWLC | SEQ ID NO:161 |
| CRGWADRKC | SEQ ID NO:162 |
| CGGWLFPPC | SEQ ID NO:163 |
| CTQVRFSGC | SEQ ID NO:164 |
| CPWWWFGEC | SEQ ID NO:165 |
| CGGWLFPPC | SEQ ID NO:166 |

### Discussion

A VEGF receptor ligand was identified with the sequence CPQPRPLC (SEQ ID NO:6) that resembles the motif PRPLC (an NRP-1 binding site found in VEGF-B₁₆₇) and the motif PQPR (embedded within a 12-residue NRP-1-binding epitope of VEGF-B₁₈₆) (Makinen et al., 1999). Thus, the VEGF-B mimetope CPQPRPLC (SEQ ID NO:6) appears to be a chimera between binding sites in different VEGF-B isoforms. These results suggest that the carboxy terminal regions of both VEGF-B isoforms may bind to and activate VEGF-R1 and NRP-1. They also suggest that the peptide CPQPRPLC (SEQ ID NO:6) may mimic the effects of both VEGF-B isoforms in its interactions with the VEGF-R1 and NRP-1 receptors. The observed differential effects on VEGF-R1 and NRP-1 using the synthetic peptide CPQPRPLC (SEQ ID NO:6) to compete with phage binding suggests that the peptide chimeric motif interacts with VEGF receptors differentially. This may be due to the number of binding sites in each receptor or the affinity of the binding sites for the chimeric peptide.

These results show that BRASIL will be of use to target cell populations derived from patient samples. The method can easily be used, for example, in tandem with fine needle aspirates of solid tumors or fluorescence activated cell sorting of white blood cells from patients with leukemia. Because unbound phage in the upper aqueous phase may be recovered with minimal losses, pre-clearing strategies are facilitated by BRASIL. This allows improved protocols for targeting peptide identification by phage display, for example by subtracting phage binding to cells from normal individuals before isolation of phage binding to diseased cells. The BRASIL method allows a decrease in non-specific background of phage binding.

Multiple samples and several rounds of pre-clearing and selection can be performed in a few hours, allowing method automation and facilitating high-throughput screening. Data (shown below) suggest that BRASIL may enable targeting of organs with a significant reticuloendothelial component such as spleen, liver, and bone marrow which has not been feasible with currently available in vivo phage display technology (Pasqualini et al., 2000). The method may also be used with phage displaying larger polypeptides or folded proteins such as enzymes or antibodies (not shown), providing a phage display based approach to high throughput screening for novel inhibitors or activators of naturally occurring enzymes, receptors or other proteins. The data show that BRASIL is superior to conventional protocols for identifying targeting ligand-receptor pairs and to probing the molecular diversity of cell surfaces.

### Example 3

### BRASIL with a Leukemia Cell Line

The BRASIL protocol has also been performed with the Molt-4 leukemia cell line and a CX5C library, using the methods described above. The library was presubtracted against a normal Molt-4 cell line and then screened against a Molt-4 cell line transformed with a gene encoding the CD-13 protein. Molt-4 leukemia targeting peptides are listed in Table 5 below.

TABLE 5

Targeting peptides against the
Molt-4 leukemia cell line

| | |
|---|---|
| CEKRWGC | SEQ ID NO:167 |
| CKQRGVC | SEQ ID NO:168 |
| CSVWFGC | SEQ ID NO:7 |
| CQVRLSC | SEQ ID NO:169 |
| CTWDKRC | SEQ ID NO:170 |
| CTLFRNC | SEQ ID NO:171 |
| CRGSAVC | SEQ ID NO:172 |
| CAISVGC | SEQ ID NO:173 |
| CTNPQRC | SEQ ID NO:174 |
| CDSWPLC | SEQ ID NO:175 |
| CENGSRC | SEQ ID NO:176 |
| CGGSSQC | SEQ ID NO:177 |
| CGRRGPC | SEQ ID NO:178 |
| CSGRSGC | SEQ ID NO:179 |
| CQQGRYC | SEQ ID NO:180 |
| CVKQMRC | SEQ ID NO:181 |
| CSVWWGC | SEQ ID NO:182 |
| CSGPC | SEQ ID NO:183 |
| CEGHQSC | SEQ ID NO:184 |
| CNVWYGC | SEQ ID NO:185 |
| CRSPMKC | SEQ ID NO:186 |
| CPTMTEC | SEQ ID NO:187 |
| CSVWFGC | SEQ ID NO:188 |
| CSVWYGC | SEQ ID NO:189 |
| CSVWYGC | SEQ ID NO:190 |
| CWILEQC | SEQ ID NO:191 |
| CMATLRC | SEQ ID NO:192 |
| CRKLGGC | SEQ ID NO:193 |
| CRAREMC | SEQ ID NO:194 |
| CQAWQRC | SEQ ID NO:195 |
| CKDRWGC | SEQ ID NO:196 |
| CYSDKKC | SEQ ID NO:197 |
| CGNHQKC | SEQ ID NO:198 |
| CPNDSLC | SEQ ID NO:199 |
| CQGTWIC | SEQ ID NO:200 |
| CMVYFGC | SEQ ID NO:8 |

A consensus sequence identified for the leukemic cell line targeting peptides was CXVWXGC (SEQ ID NO:201).

### Example 4

### Identification of Targeting Peptides for Urothelial Tissue by BRASIL

Targeting peptides for urothelial tissue have not previously been identified by phage display. The present example further demonstrates the utility of the BRASIL method for identifying novel targeting peptides and illustrates additional embodiments of the methods and compositions.

### Materials and Methods

Materials

The human cell lines T24, RT4, MDA-MB-435S, and MOLT-4 were obtained from the American Type Culture Collection (Manassas, Va.). All tissue culture media were from LifeTechnologies (NY). Cells were grown under standard conditions at 37° C. with 5% CO₂ in DMEM supplemented with 10% fetal bovine serum, 2 mM L-glutamine, 1 mM sodium pyruvate, 100 IU/ml penicillin, and 100 mg/ml streptomycin. Human urothelial cells were isolated from fresh ureter specimens and cultured using supplemented Keratinocyte SFM Medium. Pig bladders were obtained from Dr. K. Wright (Department of Veterinary Medicine, M. D. Anderson Cancer Center, Houston, Tex.). Phage display libraries were prepared and amplified with the K91kan E. coli strain as described above. Synthetic peptides were from Anaspec (San Jose, Calif.).

Panning of Phage

Urothelial cells were isolated from fresh ureter specimens of patients undergoing nephrectomy for renal cell carcinoma. Ureters were freed of connective and fat tissue, slit open and the mucosa gently scraped into PBS under sterile conditions. Cells were then pelleted and resuspended in RPMI/10% BSA. Approximately 1×10⁷ cells in 200 μl RPMI/10% BSA were then incubated with 1×10⁷ cfu of a cyclic CX₇C-phage library, a linear X₆-library or amplified phage from a previous round of bipanning for 4 hours on ice. In two separate experiments the library or amplified phage from previous rounds were precleared on 1×10⁷ MOLT-4 cells for 30 min on ice prior to adding to the normal urothelial cells. After incubation, panning was continued using the BRASIL method described above. In brief, cells were placed on an oil cushion consisting of 90% dibutylphatalate and 10% cyclohexan (Sigma, St. Louis, Mo.), in a 400 μl Eppendorf tube and pelleted for 10 min at 10,000 rpm in an Eppendorf centrifuge. The tubes were then frozen in liquid nitrogen and the lower part of the tube containing the cell pellet cut off. The pellet was reinfected with 1 ml of log-phase K91 bacteria for 1 hour after removal of excess oil and amplified over night. Small aliquots were plated out for single colony picking and sequencing. Up to 3 rounds were performed.

Sequencing and Alignments

After each round the peptide inserts of 94 randomly selected phage clones were sequenced by DNA sequencing using the primer 5′-CCCTCATAGTTAGCGTAACGATCT-3′ (SEQ ID NO:12) and the Big Dye Terminator Cycle Sequencing Kit (Perkin Elmer, Norwalk, Conn.). Peptide sequences were aligned using the ClustalW alignment program (European Bioinformatics Institute homepage, (http://www2.ebi.ac.uk/clustalw/). Enriched peptide sequences were aligned to protein databases using the BLAST program of the National Center for Biotechnology Information http://www.ncbi.nlm.nih.gov/BLAST/). Similarity was defined as percentage of positive matches in the area aligned by the program.

Phage Attachment and Competition Experiments

Binding of selected phage was examined with human adherent primary urothelial cells, the breast cancer cell line MDA-MB-435 and the transitional carcinoma cell lines RT4 and T24. All cells were grown to subconfluency in 48 well plates and free binding sites were blocked with 800 μl 30% FCS/DMEM (blocking medium) for 1 hour at 37° C. The blocking solution was then replaced by 200 μl 10% FCS/DMEM (washing medium) containing 1×10⁸ cfu of each phage per well. After incubation for 2 hours at 4° C. to prevent unspecific endocytosis, unbound phage were removed by washing 7 times with 500 μl washing medium. For competition experiments increasing concentrations of the corresponding peptide or a control peptide (CARAC, SEQ ID NO:5) were added during the incubation. Bound phage were determined by infection with 500 μl log phase K91 culture and plating of serial dilutions. Values represent means of serial dilutions of triplicates wells and are given relative to binding of insertless fd-tet phage.

To determine binding to intact mucosa a novel dot blot chamber assay was developed, placing the bladder or ureter specimen into a dot blot chamber (Biorad, Hercules, Calif.), with the mucosa facing upwards, thus generating up to 96 equally large fields of mucosa Blocking and washing in the dot blot chamber was performed as above but with 400 μl of the corresponding medium and infection was performed with 400 μl of log-phase K91kan culture per well. Three wells were pooled as one well.

Removal of the glucosaminoglycan- (GAG-) layer on intact mucosa samples was performed as a dot blot chamber assay. Half of the wells were incubated with 200 μl 0.1M HCl per well for 2 min, intensely rinsed with blocking solution and both parts blocked as before. Results are given relative to fd-tet phage to untreated mucosa, that was set to 1.

### Results

To identify peptide motifs that interact with the human bladder wall, clones were selected from phage display peptide libraries by successive rounds of affinity panning on freshly isolated urothelial cells from surgical ureter specimens. In four experiments two different libraries, a cyclic 7mer and a linear 6mer library, were panned on human urothelial cells, with or without prior subtraction against MOLT-4 leukemia cells using the BRASIL method. Up to four rounds of selection were performed and 94 clones sequenced after every round. Five peptide motifs were identified by aligning all obtained sequences with the ClustalW program (Table 6).

TABLE 6

Selection of Peptides Binding
to Human Urothelial Cells

| | | |
|---|---|---|
| | | Found |
| Peptide Sequence | Shared Motif | in Round |

| | | | |
|---|---|---|---|
| CGQEISGLC* | (SEQ ID NO:13) | ISGL | 2 |
| EVISGL | (SEQ ID NO:14) | (SEQ ID NO:35) | 1 |
| LISGVL | (SEQ ID NO:15) | | 1 |
| ELLSGL | (SEQ ID NO:16) | | 1 |
| GRLSGSL | (SEQ ID NO:17) | | 2 |
| CLRSGGLTC | (SEQ ID NO:18) | GGLS | 2 |
| LGGLSA | (SEQ ID NO:19) | (SEQ ID NO:36) | 2 |
| CWGGLSGLC* | (SEQ ID NO:20) | | 1 |
| CGLSLK | (SEQ ID NO:21) | | 1 |
| GLSARH | (SEQ ID NO:22) | | 1# (2x) |
| CEFGLSEVC | (SEQ ID NO:23) | | 1 |
| SKHALE | (SEQ ID NO:24) | HALE | 1# |
| VHALES* | (SEQ ID NO:25) | (SEQ ID NO:37) | 3 |
| VFALEG | (SEQ ID NO:26) | | 2 |
| CRIRMSAGC* | (SEQ ID NO:27) | MSAG | 1# |
| AMSAGV | (SEQ ID NO:28) | (SEQ D NO:38) | 2 |
| CMIAGLGRC | (SEQ ID NO:29) | | 1# |
| CRVIVGPRC | (SEQ ID NO:30) | RVTXG | 1# |
| CRVFAGKRC | (SEQ ID NO:31) | (SEQ ID NO:39) | 1 |
| DRVTLG* | (SEQ ID NO:32) | | 3 |
| CRVTRGHGC | (SEQ ID NO:33) | | 1# |
| CIRVEAGSC | (SEQ ID NO:34) | | 1# |

Phage selected after subtraction to MOLT-4 cells are indicated by a #.
Phage chosen for binding assays are indicated by *.

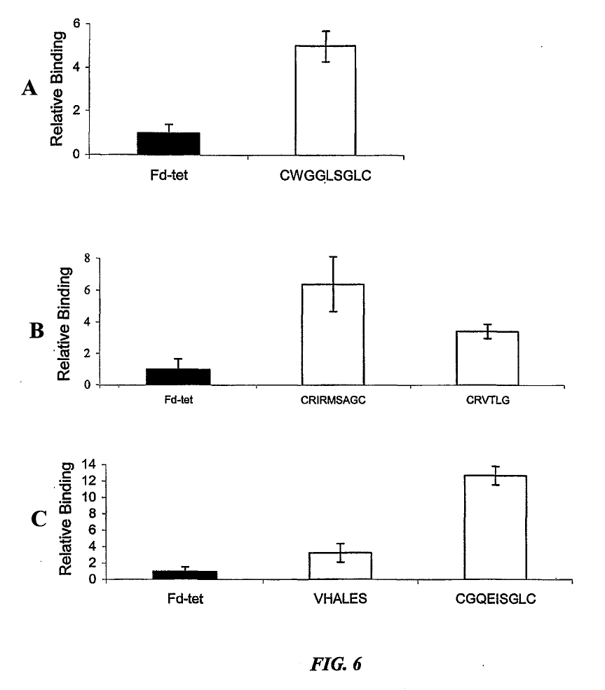

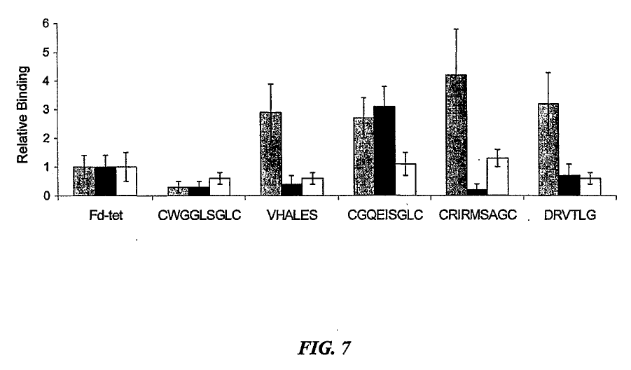

Phage containing the five peptide motifs were amplified, carefully titered and their binding to cultured human urothelial cells tested in a subconfluent monolayer. Insertless fd-tet phage were used as a negative control. Phage containing the consensus motifs bound up to 12.7 times higher to cultured urothelial cells than insertless fd-tet phage (FIG. 6). Binding was specific for urothelial cells as determined by a lack of binding to the human breast cancer cell line MDA-MB-435S, derived from a metastatic, ductal mammary carcinoma (FIG. 7).

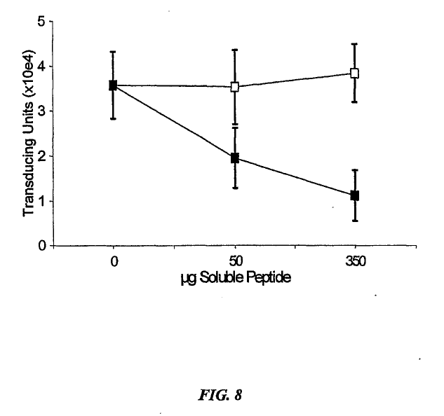

Binding to the urothelial tumor cell lines T24, derived from a poorly differentiated recurrent transitional cell carcinoma and RT4, derived from a transitional cell papilloma was also examined. All phage, except CWGGLSGLC (SEQ ID NO:20) bound to RT4, while only CGQEISGLC (SEQ ID NO:13) phage bound to T24 tumor cells (FIG. 7). VHALES (SEQ ID NO:25) phage apparently bound only to RT4 tumor cells. Binding specificity of VHALES (SEQ ID NO:25) was verified by competitive phage binding inhibition (FIG. 8). Binding was 5.4 fold higher than fd-tet phage and binding was reduced by soluble VHALES (SEQ ID NO:25) peptide in a dose-dependent manner, while remaining unchanged by equal amounts of the control peptide CARAC (SEQ ID NO:5) (FIG. 8).

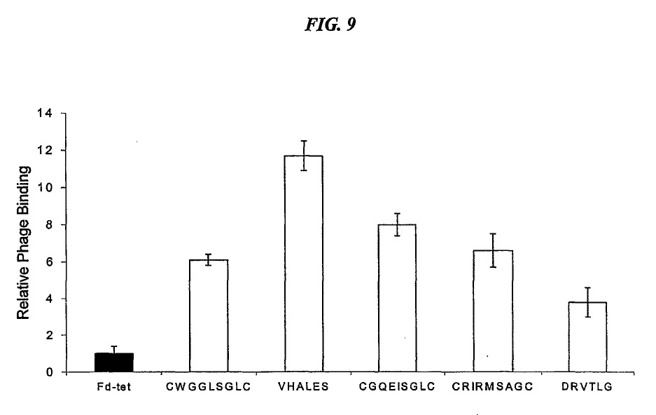

Binding of the motifs to intact bladder mucosa was examined using a novel dot blot chamber binding assay that allows the simultaneous testing of phage binding in up to 96 equal parts of bladder or ureter mucosa. Binding to intact porcine bladder and human ureter mucosa was determined with this assay. Selected phage displaying a consensus motif bound 3.8 to 11.7 times higher to porcine bladder mucosa than fd-tet phage (FIG. 9).

Using the same assay the influence of the glucosaminoglycan (GAG) layer on binding of several phage to intact mucosa was examined. The GAG-layer was removed as described above. Half of the mucosa of human ureter and porcine bladder specimens were treated inside the dot blot chamber with 0 μM HCl for 2 min, extensively washed and the binding assay performed as before.

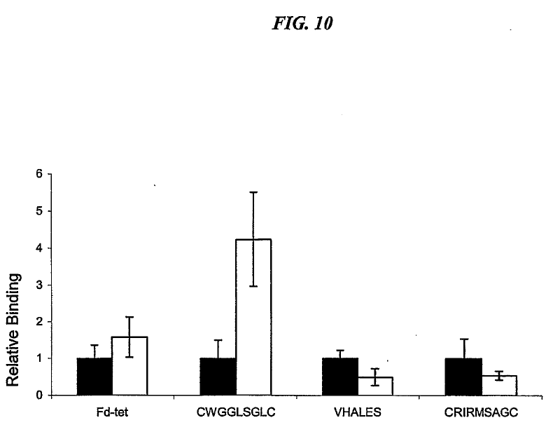

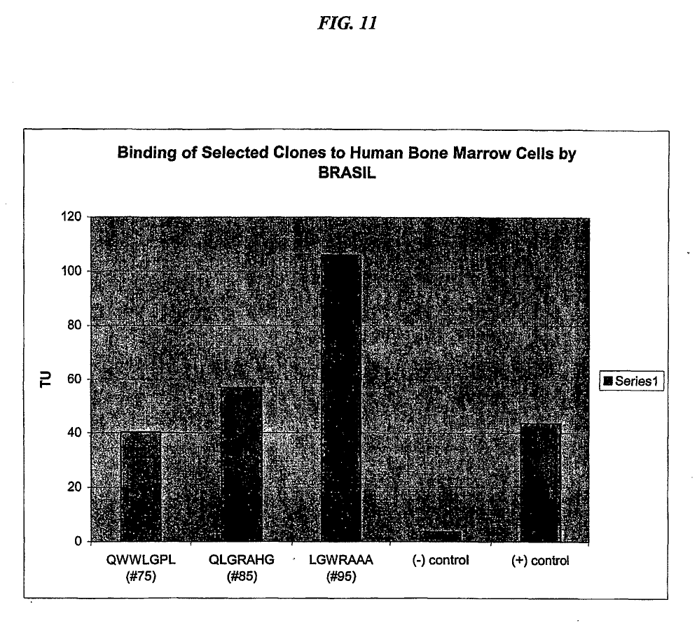

Binding of phage after GAG-removal was compared relative to that of untreated mucosa, which was set to 1. The binding of the control phage fd-tet and CRIRMSAGC (SEQ ID NO:27) phage remained unchanged by the treatment, while CWGGLSGLC (SEQ ID NO:20) phage binding increased 4.2 fold. VHALES (SEQ ID NO:25) phage binding was reduced by 50% (FIG. 10). When tested on a human ureter sample GAG-removal increased CWGGLSGLC phage binding 3.4 fold (data not shown). This suggests a negative influence of the GAG-layer on binding of CWGGLSGLC (SEQ ID NO:20) phage.

These results show a number of new targeting peptide sequences and conserved motifs targeted to urothelial tissues. Further modifications of the BRASIL protocol are demonstrated herein, along with the utility of those novel methods for identification and characterization of targeting peptide sequences. The skilled artisan will realize that the disclosed methods and compositions are not limited to urothelial cells or tissues, but rather have broad applicability to a variety of organs, tissues and cell types found in humans.

### Example 5

### BRASIL and Stem Cell Screening

Another non-limiting example of cell types that may be screened for targeting peptide sequences by BRASIL includes stem cells. In the discussion below, the stem cells are obtained from bone marrow. However, the skilled artisan will realize that the disclosed methods are applicable for stem cells in general.

Source of Cells and Culture

Mesenchymal cells are primary stem cells derived from bone marrow, obtained by seeding human bone marrow aspirate onto plastic flasks. Cells that attach to the flask are the mesenchymal cells. Mesenchymal cells were cultured in RPMI 1640 medium supplemented with 20% fetal calf serum at 37° C. (5% CO₂) and sub-cultured every 4-5 days. KS1767 cells were grown in MEM medium supplemented with 10% fetal calf serum and sub-cultured every 3-5 days.

Biopanning on Mesenchymal Cells

A subtraction strategy was performed in which the phage library was first prescreened against KS1767 cells and phage binding to the KS1767 non-stem cell line were removed. The pre-screened library was then screened against mesenchymal cells using the BRAZIL method.

All procedures were performed at 4° C. All media and solutions used for the biopanning were filtered through a 0.22 μm Millipore filter. The mesenchymal and KS1767 cells were washed with PBS and incubated with PBS plus 5 mM EDTA for 10 minutes on ice to promote detachment of the cells from the plastic. Cells were collected by aspirating the medium, washed by centrifugation with RPMI 1640 medium and re-suspended at 10⁶ cells/ml in RPMI 1640 medium supplemented with 0.5% BSA (bovine serum albumin). A CX7C phage display library (or phage obtained from the previous round of biopanning) was added to the KS cells (10⁹ T.U. of phage per 10⁵ cells) and incubated for 1-2 h on ice. Unbound phage were selected by BRASIL after KS1767 cells were exposed to phage and centrifuged over dibutyl phthalate:cyclohexane (6:1) at 4° C. Under these conditions, cells carrying bound phage pellet at the bottom of the tube. Unbound phage remain in the upper (aqueous) phase. The upper phase was carefully transferred to a new tube containing 105 mesenchymal cells and further incubated for 4 h on ice. Bound phage attached to mesenchymal cells were then selected by BRASIL. The mesenchymal cell suspension with phage was centrifuged over dibutyl phthalate:cyclohexane (6:1) at 4° C. Mesenchymal cells carrying the bound phage pelleted at the bottom of the tube. The tube was snap frozen at −80° C. for 10 minutes and the bottom of the tube containing the pellet of cells with bound phage was cut off, transferred to a new tube and the pellet carefully ressuspended with 200 μl of a log-phase E. coli K91 culture to recover the phage. After 20 minutes of infection, 20 ml of LB medium (Luria-Bertani) was added and the cells cultured for 16-18 h at 37° C. with agitation for phage amplification. After the initial selection, phage obtained from a previous round was used for the next round of selection.

After 3 rounds of biopanning, individual colonies were selected for sequencing. The stem cell binding peptides are listed in Table 7 below.

TABLE 7
Stem cell (mesenchymal) targeting peptides

| | |
|---|---|
| CLGRLTVLC | (SEQ ID NO:63) |
| CTAWFIESC | (SEQ ID NO:64) |
| CSYGRASLC | (SEQ ID NO:65) |
| CDAGPWTAC | (SEQ ID NO:66) |
| CVGVGRSRC | (SEQ ID NO:67) |
| CTNPWSPVC | (SEQ ID NO:68) |
| CGGSYDEVC | (SEQ ID NO:69) |
| CAPMEWSVC | (SEQ ID NO:70) |
| CTRVHGLAC | (SEQ ID NO:71) |
| CESLSHVDC | (SEQ ID NO:72) |
| CLWTQSSGC | (SEQ ID NO:73) |
| CERSIGFAC | (SEQ ID NO:74) |
| CSVPVSSSC | (SEQ ID NO:75): |
| CYPGYDSYC | (SEQ ID NO:76) |
| CPWYWFGTC | (SEQ ID NO:77) |
| CECRGDCYC | (SEQ ID NO:78) |
| CVKKGGFWC | (SEQ ID NO:79) |
| CSMTKLGAC | (SEQ ID NO:80) |
| CGVLKPYLC | (SEQ ID NO:81) |
| CWWPWGWGC | (SEQ ID NO:94) |
| CSWWTFGFC | (SEQ ID NO:95) |
| CNSRAGSVC | (SEQ ID NO:96) |
| CLRLSMSAC | (SEQ ID NO:97) |
| CNSRAGSVC | (SEQ ID NO:98) |
| CMSGNTERC | (SEQ ID NO:99) |
| CGHLGSVYC | (SEQ ID NO:100) |
| CVLADPTGC | (SEQ ID NO:101) |
| CECRGDCYC | (SEQ ID NO:102) |
| CWWGWWGTC | (SEQ ID NO:103) |
| CWKGFGWWC | (SEQ ID NO:104) |
| CKRSATILC | (SEQ ID NO:105) |
| CIEGRRGLC | (SEQ ID NO:106) |
| CPWYWLGWC | (SEQ ID NO:107) |
| CVRQGEDAC | (SEQ ID NO:108) |
| CSLAVPLAC | (SEQ ID NO:109) |
| CMMHGLAAC | (SEQ ID NO:110) |
| CDWWTTAWC | (SEQ ID NO:111) |
| CGWWGLWPC | (SEQ ID NO:112) |
| CPWYWFGTC | (SEQ ID NO:82) |
| CWVADGYRC | (SEQ ID NO:83) |
| CECRGDCYC | (SEQ ID NO:84) |
| CSHAVMPWC | (SEQ ID NO:85) |
| CSERIARVC | (SEQ ID NO:86) |
| CPWYWLGWC | (SEQ ID NO:87) |
| CGRKNEWAC | (SEQ ID NO:88) |
| CARDRIIAC | (SEQ ID NO:89) |
| CGQMNREVC | (SEQ ID NO:90) |
| CDAYPLFFC | (SEQ ID NO:91) |
| CWKGFGWWC | (SEQ ID NO:92) |
| CLGSGSGSC | (SEQ ID NO:93) |
| CGWFSWFGC | (SEQ ID NO:113) |
| CRVDFSKGC | (SEQ ID NO:114) |
| CSSLATVVC | (SEQ ID NO:115) |
| CMYRTSLAC | (SEQ ID NO:116) |
| CLAAVYQSC | (SEQ ID NO:117) |
| CSRRVIGAC | (SEQ ID NO:118) |
| CSWWNWFGC | (SEQ ID NO:119) |
| CSRRPEVVC | (SEQ ID NO:120) |
| CVTGNRGC | (SEQ ID NO:121) |
| CVSWWFWGC | (SEQ ID NO:122) |
| CGWFSWWGC | (SEQ ID NO:123) |
| CSWWRFGYC | (SEQ ID NO:124) |

Receptor Identification

The phage “D5” containing the peptide sequence CRVDFSKGC (SEQ ID NO:114) showed significant homology with the leptin hormone (Table 8). This region of leptin is conserved in several species (Macaca mulatta, Homo sapiens, Pan troglodytes, Gorilla gorilla, Pongo pygmaeus, Mus musculus, Rattus nervegicus).

TABLE 8
Homology between phage D5 and leptin sequences

| | | |
|---|---|---|
| Phage | 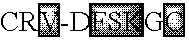 | (SEQ ID NO:114) |
| Human leptin | 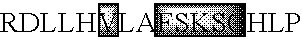 | (SEQ ID NO:125) |
| Mouse leptin | 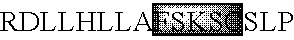 | (SEQ ID NO:126) |

The conserved peptide maps to a loop in between amino acids 90-96 in the protein (Zhang et al, 1997). This region of the leptin molecule has been indicated as important for leptin activity. A synthetic peptide DLLHLLAFSKSCSLLP (SEQ ID NO:127) has been reported to block leptin activity in vivo (Grasso et al., 1997) (amino acids in bold indicate those with similarity to clone D5 (CRVDFSKGC, SEQ ID NO:114).

The present example shows that BRASIL can be used to identify targeting peptides against stem cells. The homology between one of the identified peptide sequences and an endogenous hormone further validates the identified sequences. The skilled artisan will realize that the methods and targeting peptide sequences identified herein are of potential use for identification and purification of stem cells (for example, by affinity chromatography) and for identification of receptor:ligand pairs present in stem cells.

### Example 6

### Bone Marrow Screening by BRASIL

A non-limiting example of an organ of specific interest for targeting peptides is bone marrow. Bone is the preferred site of metastasis in the large majority of patients with prostate cancer (Fidler, 1999). This striking selectivity has been viewed as an example of site-specific interactions that were essential to cancer progression (Rak, 1995; Zetter, 1998). Despite the clinical relevance, little is known about the mechanisms that control prostate cancer spread to bone. In addition, there were no effective strategies for targeting therapeutic agents for the treatment of metastatic prostate cancer (Brodt et. al, 1996).

A subset of peptides capable of selective homing to bone marrow through the circulation is likely to simulate the behavior of prostate cancer cells during bone metastasis formation. The vascular markers targeted by using phage display might also be utilized by tumor cells to metastasize. This concept has already been proven to be true for lung-homing peptides. Peptides that home to lung blood vessels inhibit experimental metastasis. These results fit a “modified seed and soil” model, in which the basis for site-specific metastasis is the presence of homing receptors in blood vessels of certain tissues to which metastasis preferentially occurs. Such selective vascular markers are exposed to tumor cells during adhesion, the first step of the metastatic cascade. Isolation of bone marrow-homing peptides is of utility for identifying those vascular markers that mediate prostate cancer cell homing during the metastatic process, and for potential therapeutic intervention in preventing metastases to bone, or in selectively imaging and/or treating cancer that has already metastasized to bone.

Screening of Phage Display Libraries on Human Bone Marrow:

Fresh human ribs removed during surgery for access to underlying tumors were sectioned to expose the bone marrow surface. No significant damage to the bone marrow was inflicted to the tissue and the morphology was well preserved during after the procedure. The bone samples were washed (gently) 5 times with ice cold DMEM/0.15% BSA (sterile filtered). The marrow was removed by gently scraping cells from the bone. Cells were washed twice by centrifugation and resuspension in DMEM/BSA to remove debris and fat. Cells were resuspended in DMEM/BSA (about 10⁷ cells per ml) and incubated with a phage display library (10⁹ TU) prepared as described above. After incubation for 3 hours on ice, the cells were centrifuged through an organic phase consisting of a 9:1 mixture of dibutylphthalate:cyclohexane. Centrifugation occurred for 10 min at 10,000×g. The bottom of the centrifuge tube was snap frozen at −80° C. and phage were recovered by bacterial infection as described above. The selection was repeated for 3 more rounds of BRASIL and 90 clones were sequenced. The bone marrow targeting sequences are listed in Table 9 below.

TABLE 9

Bone Marrow Targeting Peptides
Identified by BRASIL

| | |
|---|---|
| CPEVMGSSC | SEQ ID NO:202 |
| CSSVVRLGC | SEQ ID NO:203 |
| CVGAGLHIC | SEQ ID NO:204 |
| CHLEPDWVC | SEQ ID NO:205 |
| CALGRWDRC | SEQ ID NO:206 |
| CFGGVGSWC | SEQ ID NO:207 |
| CGRRDTVDC | SEQ ID NO:208 |
| CLVLGGYGC | SEQ ID NO:209 |
| CWENRGQFC | SEQ ID NO:210 |
| CREQASTGC | SEQ ID NO:211 |
| CVVKLRNRC | SEQ ID NO:212 |
| CVGLRAPLC | SEQ ID NO:213 |
| CQKVARPGC | SEQ ID NO:214 |
| CQKFARPGC | SEQ ID NO:215 |
| CMWGLSYLC | SEQ ID NO:216 |
| CREQRHNLC | SEQ ID NO:217 |
| CLVLSASAC | SEQ ID NO:218 |
| CLLSGLMGC | SEQ ID NO:219 |
| CRGDTKALC | SEQ ID NO:220 |
| CVSQLGRVC | SEQ ID NO:221 |
| CFVFEAMGC | SEQ ID NO:222 |
| CSVIKRGAC | SEQ ID NO:223 |
| CGGWVDHRC | SEQ ID NO:224 |
| CAVVRNQEC | SEQ ID NO:225 |
| CDSPRRPVC | SEQ ID NO:226 |
| CTFSGHRLC | SEQ ID NO:227 |
| CHTWGGRNC | SEQ ID NO:228 |
| CEGAGLVAC | SEQ ID NO:229 |
| CFPRVWSRC | SEQ ID NO:230 |
| CYWLGGALC | SEQ ID NO:231 |
| CDTNQRVVC | SEQ ID NO:232 |
| CMRVTKTHC | SEQ ID NO:233 |
| CDQNWLVHC | SEQ ID NO:234 |
| CTFSGHRLC | SEQ ID NO:235 |
| CALSAYRVC | SEQ ID NO:236 |
| CGGEEGRRC | SEQ ID NO:237 |
| CAEAGGPDC | SEQ ID NO:238 |
| CIVMLGWRC | SEQ ID NO:239 |
| CGHGVTGRC | SEQ ID NO:240 |
| CERGRGAAC | SEQ ID NO:241 |
| CAAGEGWWC | SEQ ID NO:242 |
| CALSAYRVC | SEQ ID NO:243 |
| CLWPWAGEC | SEQ ID NO:244 |
| CTHATWLVC | SEQ ID NO:245 |
| CSGVSTVRC | SEQ ID NO:246 |
| CLVSYMNGC | SEQ ID NO:247 |
| CVRTSSQWC | SEQ ID NO:248 |
| CLGKGLSSC | SEQ ID NO:249 |
| CFTAVEQGC | SEQ ID NO:250 |
| CGGIGPRFC | SEQ ID NO:251 |
| CVATWCEKC | SEQ ID NO:252 |
| CSSELRAAC | SEQ ID NO:253 |
| CKGSLDEIC | SEQ ID NO:254 |
| CSSVVRLGC | SEQ ID NO:255 |
| CLKTEFTAC | SEQ ID NO:256 |
| CPGRLWRAC | SEQ ID NO:257 |
| CSELGGAGC | SEQ ID NO:258 |
| CLGWRAAAC | SEQ ID NO:259 |
| CGAMWGMGC | SEQ ID NO:260 |
| CIGLSGIEC | SEQ ID NO:261 |
| CQKLGWRV | SEQ ID NO:262 |
| CLEWLQQVC | SEQ ID NO:263 |
| CLVLGEKPC | SEQ ID NO:264 |
| CAAGKGLLC | SEQ ID NO:265 |
| CAAGKDLLC | SEQ ID NO:266 |
| CGAQSPRC | SEQ ID NO:267 |
| CLSSVRGWC | SEQ ID NO:268 |
| CSESQLAWC | SEQ ID NO:269 |
| CSRNSVREC | SEQ ID NO:270 |
| CGLVITATC | SEQ ID NO:271 |
| CPGSVRVQC | SEQ ID NO:272 |
| CRGDTKALC | SEQ ID NO:273 |
| CACVRSRNC | SEQ ID NO:274 |
| CRADSEGVC | SEQ ID NO:275 |
| CNVEASVRC | SEQ ID NO:276 |
| CVGNAKLMC | SEQ ID NO:277 |
| CQKLARAGC | SEQ ID NO:278 |
| CGGRAILLC | SEQ ID NO:279 |
| CQLGRAHGC | SEQ ID NO:280 |
| CGLVITATC | SEQ ID NO:281 |
| CVGATYSRC | SEQ ID NO:282 |
| CSAFSVAYC | SEQ ID NO:283 |
| CLAWEVYLC | SEQ ID NO:284 |
| CQWWLGPLC | SEQ ID NO:285 |
| CSLGSFMGC | SEQ ID NO:286 |
| CVLGEISWC | SEQ ID NO:287 |
| CSGGSGARC | SEQ ID NO:288 |
| CPWWMMERC | SEQ ID NO:289 |

Statistical Analysis of the Peptide Motifs

A system has been designed to analyze the data resulting from peptide library screenings, adapted from the SAS package. The system is available upon request from the M. D. Anderson Cancer Center. Based on a statistical analysis of the phage sequences listed in Table 9, an LG motif (Leu-Gly) was observed in bone marrow targeting phage. Selected clones with the motif showed very high binding to human bone marrow cells compared to the negative control (insertless fd-tet phage). The positive control was phage containing an RGD-4C insert, which is known to bind to bone marrow. The highest affinity peptide (CLGWRAAAC, SEQ ID NO:259) exhibited binding that was over twice as high as the positive control. Binding assays were performed using BRASIL as described above, except that a single phage clone was used in place of the phage library.

The skilled artisan will realize that the bone marrow targeting peptide sequences identified herein will be of use for numerous applications within the scope of the present invention, including but not limited to targeted delivery of therapeutic agents or gene therapy, in vivo imaging of normal or diseased organs or tissues, identification of receptors and receptor ligands in organs or tissues, and therapeutic treatment of a number of human diseases, particularly metastatic prostate cancer.

All of the COMPOSITIONS, METHODS and APPARATUS disclosed and claimed herein can be made and executed without undue experimentation in light of the present disclosure. While the compositions and methods of this invention have been described in terms of preferred embodiments, it are apparent to those of skill in the art that variations may be applied to the COMPOSITIONS, METHODS and APPARATUS and in the steps or in the sequence of steps of the methods described herein without departing from the concept, spirit and scope of the invention. More specifically, it are apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Arap, W., Pasqualini R., and Ruoslahti, E. Cancer treatment by targeted drug delivery to tumor vasculature. Science 279:377-380, 1998a.
Arap W, Pasqualini R, and Ruoslahti E. Chemotherapy targeted to tumor vasculature. Curr. Opin. Oncol., 1998b.
Baichwal and Sugden, In: Gene Transfer, Kucherlapati R, ed., New York, Plenum Press, pp. 117-148, 1986.
Barany and Merrifield, The Peptides, Gross and Meienhofer, eds., Academic Press, New York, pp. 1-284, 1979.
Brodt et. al, The role of marrow endothelium in the localization of metastatic cancer cells to bone. In Bone Metastasis—mechanisms and pathophysiology, pp 17-23, 1996. (Orr and Singh, eds.)
Brooks P C, Clark R A, Cheresh D A. Requirement of vascular integrin αvβ3 for angiogenesis. Science 264:569-571, 1994a.
Brooks, P. C., Montgomery A. M., Rosenfeld, M., Reisfeld, R. A., Hu, T., Klier, G., and Cheresh D. A. Integrin αvβ3 antagonists promote tumor regression by inducing apoptosis of angiogenic blood vessels. Cell 79, 1157-1164, 1994b
Burg M, Pasqualini R, Arap W, Stallcup W, and Ruoslahti E. Identification of NG2 proteoglycan-binding peptides that home to tumor neovasculature. Cancer Res 58:2869-2874, 1999.
Burg, M. A., Pasqualini, R., Arap, W., Ruoslahti, E. & Stallcup, W. B. NG2 proteoglycan-binding peptides target tumor neovasculature. Cancer Res 59, 2869-2874, 1999.
Chen and Okayama, Mol. Cell. Biol., 7:2745-2752, 1987.
Coffin, In: Virology, Fields et al., eds., Raven Press, New York, pp. 1437-1500, 1990.
Couch et al., Am. Rev. Resp. Dis., 88:394-403, 1963.
Coupar et al., Gene, 68:1-10, 1988.
Eisen, T. et al. Continuous low dose Thalidomide: a phase II study in advanced melanoma, renal cell, ovarian and breast cancer. Br J Cancer 82, 812-817, 2000.
Ellerby H M, Arap W, Ellerby L, Kain R, Andrusiak R, Rio G, Krajeswli S, Lombardo C, Rao R, Ruoslahti E, Bredesen D, and Pasqualini R. Anti-cancer Activity of Targeted proapoptotic peptides. Nature Med 9:1032-1038, 1999
Engerman, R. L. and Kern, T. S. (1986) Hyperglycemia as a cause of diabetic retinopathy. Metabolism 35(S1), 20-23.
Ferrara, N. and Davis-Smyth, T. (1997) The biology of vascular endothelial growth factor. Endocr. Rev., 18, 4-25.
Folkman J. Angiogenesis in cancer, vascular, rheumatoid and other disease. Nature Med 1:27-31, 1995
Folkman J. Addressing tumor blood vessels. Nature Biotechnol. 15: 510, 1997.
Friedmann, Science, 244:1275-1281, 1989.
Ghosh-Choudhury et al., EMBO J., 6:1733-1739, 1987.
Gomez-Foix et al., J. Biol. Chem., 267:25129-25134, 1992.
Graham and Van Der Eb, Virology, 52:456-467, 1973.
Graham and Prevec, In: Methods in Molecular Biology: Gene Transfer and Expression Protocol, E. J. Murray, ed., Humana Press, Clifton, N.J., 7:109-128, 1991.
Graham et al., J. Gen. Virol., 36:59-72, 1977.
Gopal, Mol. Cell. Biol., 5:1188-1190, 1985.
Grasso, P., Leinung, M. C., Ingher, S. P. and Lee, D. W. (1997) Endocrinology, 138(4), 1413-18.
Grunhaus and Horwitz, Seminar in Virology, 3:237-252, 1992.
Hammes H P, Brownlee M, Jonczyk A, Sutter A, and Preissner K T. Subcutaneous injection of a cyclic peptide antagonist of vitronectin receptor-type integrins inhibits retinal neovascularization. Nature Med. 2: 529-533, 1996.
Hanahan, D. and Folkman, J. (1996) Patterns and Emerging Mechanisms of the Angiogenic Switch during Tumorogenesis. Cell, 86, 353-364.
Hansen, A. S., Norén, O., Sjöström, H. and Wedelin, O. (1993) A mouse aminopeptidase-N is a marker for antigen presenting cells and appears to be co-expressed with major histocompatibility complex class II molecules. Eur. J. Immunol., 23, 2358-64.
HARLOW, E., and LANE, D. (1988). Antibodies: A Laboratory Manual (Cold Spring Harbor Laboratory Press, New York, N.Y.).
Hermonat and Muzycska, Proc. Natl. Acad. Sci. USA, 81:6466-6470, 1984.
Hersdorffer et al., DNA Cell Biol., 9:713-723, 1990.
Herz and Gerard, Proc. Natl. Acad. Sci. USA, 90:2812-2816, 1993.
Horwich, et al., J. Virol., 64:642-650, 1990.
Johnson et al., “Peptide Turn Mimetics” in BIOTECHNOLOGY AND PHARMACY, Pezzuto et al., Eds., Chapman and Hall, New York (1993).
Jones and Shenk, Cell, 13:181-188, 1978.
Karlsson et al., EMBO J., 5:2377-2385, 1986.
Koivunen E, Arap W, Valtanen H, Rainisalo A, Gahmberg C G, Salo T, Konttinen Y T, Sorsa T, Ruoslahti E, Pasqualini R. Tumor targeting with a selective gelatinase inhibitor. Nature Biotechnol 17:768-774, 1999
Kong H L and Crystal R G. Gene therapy strategies for tumor antiangiogenesis. J. Natl. Cancer Inst. 90:273-286, 1998.
KOZARSKY, K., JOOSS, K., DUNAHEE, M., STRAUSS, J E., and WILSON, J. M. (1996). Effective treatment of familial hypercholesterolaemia in the mouse model using adenovirus-mediated transfer of the VLDL receptor gene. Nat. Genet. 13; 54-62.
Le Gal La Salle et al., Science, 259:988-990, 1993.
Levrero et al., Gene, 101:195-202, 1991.
Look A T, Ashmun R A, Shapiro L H and Peiper S C. Human myeloid plasma membrane glycoprotein CD13 (gp150) is identical to aminopeptidase N.J. Clin. Invest. 83:1299-1307, 1989.
Mann et al., Cell, 33:153-159, 1983.
Markowitz et al., J. Virol., 62:1120-1124, 1988.
Merrifield, Science, 232: 341-347, 1986
Mulligan, Science, 260:926-932, 1993.
Mustonen T and Alitalo K. Endothelial receptor tyrosine kinases involved in angiogenesis. J. Cell Biol. 129:895-898, 1995.
Nicolas and Rubinstein, In: Vectors: A survey of molecular cloning vectors and their uses, Rodriguez and Denhardt, eds., Stoneham: Butterworth, pp. 494-513, 1988.
Nicolau et al., Methods Enzymol., 149:157-176, 1987.
Olofsson, B., Pajusola, K., Kaipainen, A., Euler, G., Joukov, V., Saksela, O., Orpana, A., Pettersson, R. F., Alitalo, K. and Eriksson, U. (1996) Vascular Endothelial Growth factor B, a novel growth factor for endothelial cells. Proc Natl Acad Sci USA, 93, 2576-2581.
Olofsson, B. Jeltsch, M., Eriksson, U. and Alitalo, K (1999) Current Biology of VEGF-B and VEGF-C. Curr Op Biotechnol, 10, 528-535.
Paskind et al., Virology, 67:242-248, 1975.
Pasqualini, R. and Hemler, M. E. Contrasting roles for integrin b1 and b5 cytoplasmic domains in subcellular localization, cell proliferation, and cell migration. J. Cell Biol. 125:447-60, 1994.
Pasqualini R and Ruoslahti E. Organ targeting in vivo using phage display peptide libraries. Nature 380:364-366, 1996.
Pasqualini R, Koivunen E, and Ruoslahti E. A peptide isolated from phage display libraries is a structural and functional mimic of an RGD-binding site on integrins. J. Cell Biol. 130:1189-1196, 1995.
Pasqualini R, Koivunen E, and Ruoslahti E. αv integrins as receptors for tumor targeting by circulating ligands. Nature Biotechnol 15:542-546, 1997
Pasqualini, R. Vascular Targeting with Phage Display Peptide Libraries. The Quart. J. Nucl. Med. 43:159-162, 1999.
Pasqualini, R., Arap W., Koivunen, E., Kain, R., Lahdenranta, J., Shapiro, L., Sakamoto, M., Stryn, A. and Ruoslahti, E. Aminopeptidase N is a receptor for tumor-homing peptides and a target for inhibiting angiogenesis. Cancer Res. 60: 722-727, 2000.
Potter et al., Proc. Nat. Acad. Sci. USA, 81:7161-7165, 1984.
Racher et al., Biotechnology Techniques, 9:169-174, 1995.
Ragot et al., Nature, 361:647-650, 1993.
Rajotte D and Ruoslahti E. Membrane dipeptidase is the receptor for a lung-targeting peptide identified by in vivo phage display. J Biol Chem 274:11593-11598, 1999
Rajotte D, Arap W, Hagedorn M, Koivunen E, Pasqualini R, and Ruoslahti E. Molecular heterogeneity of the vascular endothelium revealed by in vivo phage display. J Clin Invest 102:430-437, 1998
Rak J W, St. Croix B D, and Kerbel R S. Consequences of angiogenesis for tumor progression, metastasis and cancer. Anticancer Drugs 6:3-18, 1995. Remington's Pharmaceutical Sciences, 15th ed., pp. 1035-1038 and 1570-1580.
Renan, Radiother. Oncol., 19:197-218, 1990.
Rich et al., Hum. Gene Ther., 4:461-476, 1993.
Ridgeway, In: Vectors: A Survey of Molecular Cloning Vectors and Their Uses, Rodriguez et al., eds., Stoneham: Butterworth, pp. 467-492, 1988.
Rippe et al., Mol. Cell. Biol., 10:689-695, 1990.
Rosenfeld et al., Cell, 68:143-155, 1992.
Rosenfeld et al., Science, 252:431-434, 1991.
Ruoslahti E. RGD and other sequence recognition sequences for integrins. Annu. Rev. Cell Dev. Biol. 12:697-715, 1996
Smith, G. P. 1985. Filamentous fusion phage: novel expression vectors that display cloned antigens on the virion surface. Science 228:1315-7.
Smith G P, and Scott J K. Searching for peptide ligands with an epitope library. Science 228:1315-1317, 1985
Smith G P, and Scott J K. Libraries of peptides and proteins displayed in filamentous phage. Meth. Enzymol. 21:228-257, 1993.
Stewart and Young, Solid Phase Peptide Synthesis, 2d. ed., Pierce Chemical Co., 1984.
Stratford-Perricaudet and Perricaudet, In: Human Gene Transfer, O. Cohen-Haguenauer et al., eds., John Libbey Eurotext, France, pp. 51-61, 1991.
Stratford-Perricaudet et al, Hum. Gene. Ther., 1:241-256, 1990.
Tam et al., J. Am. Chem. Soc., 105:6442, 1983.
Temin, In: Gene Transfer, Kucherlapati R, ed., New York, Plenum Press, pp. 149-188, 1986.
Tischer, E., Mitchell, R., Hartman, T., Silvia, M., Gospodarowicz, D., Fiddes, J. C. and Abraham, J. (1991) the human Gene for Vascular Endothelial Growth Factor. J. Biol. Chem., 226, 11947-11954.
Top et al., J. Infect. Dis., 124:155-160, 1971.
Tur-Kaspa et al., Mol. Cell. Biol., 6:716-718, 1986.
U.S. Pat. No. 3,652,761
U.S. Pat. No. 3,817,837
U.S. Pat. No. 3,850,752
U.S. Pat. No. 3,939,350
U.S. Pat. No. 3,933,997
U.S. Pat. No. 3,996,345
U.S. Pat. No. 4,267,234
U.S. Pat. No. 4,275,149
U.S. Pat. No. 4,277,437
U.S. Pat. No. 4,366,241
U.S. Pat. No. 4,599,307
U.S. Pat. No. 4,472,509
U.S. Pat. No. 4,704,891
U.S. Pat. No. 4,727,020
U.S. Pat. No. 5,021,236
U.S. Pat. No. 5,206,347
U.S. Pat. No. 5,223,409
U.S. Pat. No. 5,401,511
U.S. Pat. No. 5,492,807
U.S. Pat. No. 5,603,872
U.S. Pat. No. 5,622,699
U.S. Pat. No. 5,670,312
U.S. Pat. No. 5,705,610
U.S. Pat. No. 5,840,841
U.S. Pat. No. 5,853,984
U.S. Pat. No. 5,889,155
U.S. Pat. No. 5,948,627
U.S. Pat. No. 6,068,829
Varmus et al., Cell, 25:23-36, 1981.
Veikkola, T. and Alitalo, K. (1999) VEGFs, receptors and angiogenesis. Seminar Cancer Biol., 9, 211-220.
Watson C A, Camera-Benson L, Palmer-Croker R and Pober J S. Variability among human umbilical vein endothelial cell cultures. Science 268: 447-448, 1995.
Wickham T J. Haskard D. Segal D. Kovesdi I. Targeting endothelium for gene therapy via receptors upregulated during angiogenesis and inflammation. Cancer Immunol. Immunother. 45:149-151, 1997c.
Wong et al., Gene, 10:87-94, 1980.
Wu and Wu, Biochemistry, 27: 887-892, 1988.
Wu and Wu, J. Biol. Chem., 262: 4429-4432, 1987.
Zetter B R. Angiogenesis and tumor metastasis. Ann Rev Med 49:407-424, 1998
Zhang et al., Nature 387:206-9, 1997.

1-87. (canceled)

88. A peptide of 10 amino acids or less in size, comprising a VEGFR-1 targeting sequence having at least three contiguous amino acids of CPQPRPLC (SEQ ID NO:6), wherein the peptide selectively binds to VEGFR-1.

89. The peptide of claim 88, wherein said peptide is 7 amino acids or less in size.

90. The peptide of claim 89, wherein said peptide is 5 amino acids or less in size.

91. The peptide of claim 90, wherein said peptide is 3 amino acids in size.

92. The peptide of claim 88, wherein the VEGFR-1 targeting sequence is PRPLC.

93. The peptide of claim 88, wherein the three contiguous amino acids are PQP.

94. The peptide of claim 88, wherein the three contiguous amino acids are QPR.

95. The peptide of claim 88, wherein the three contiguous amino acids are PRP.

96. The peptide of claim 88, wherein the three contiguous amino acids are RPL.

97. The peptide of claim 88, further defined as a cyclic peptide.

98. The peptide of claim 88, wherein said peptide is attached to a molecule.

99. The peptide of claim 98, wherein the molecule is a protein and the peptide is conjugated to the protein to form a protein conjugate.

100. The peptide of claim 99, wherein the peptide is positioned at the N or C terminus of the protein.

101. The peptide of claim 98, wherein said molecule is a drug, a chemotherapeutic agent, a radioisotope, a pro-apoptosis agent, an anti-angiogenic agent, a hormone, a cytokine, a growth factor, a cytotoxic agent, a peptide, a protein, an antibiotic, an antibody, a Fab fragment of an antibody, an imaging agent, a nucleic acid or an antigen.

102. The peptide of claim 101, wherein said molecule is a pro-apoptosis agent selected from the group consisting of gramicidin, magainin, mellitin, defensin, cecropin, (KLAKLAK).sub.2 (SEQ ID NO:1), (KLAKKLA).sub.2 (SEQ ID NO:2), (KAAKKAA).sub.2 (SEQ ID NO:3) or (KLGKKLG).sub.3 (SEQ ID NO:4).

103. The peptide of claim 101, wherein said molecule is an anti-angiogenic agent selected from the group consisting of thrombospondin, angiostatin, endostatin or pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-.beta., 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, paclitaxel, accutin, cidofovir, vincristine, bleomycin, AGM-1470, platelet factor 4 or minocycline.

104. The peptide of claim 101, wherein said molecule is a cytokine selected from the group consisting of interleukin 1 (IL-1), IL-2, IL-5, IL-10, IL-11, IL-12, IL-18, interferon-γ (IF-γ), IF-α, IF-β, a tumor necrosis factor, and GM-CSF (granulocyte macrophage colony stimulating factor).

105. The peptide of claim 98, wherein said peptide is attached to a macromolecular complex.

106. The peptide of claim 105, wherein said complex is a virus, a bacteriophage, a bacterium, a liposome, a microparticle, a magnetic bead, a yeast cell, or a mammalian cell.

107. The peptide of claim 106, wherein said peptide is attached to a virus.

108. The peptide of claim 107, wherein said virus is a papoavirus, adenovirus, retrovirus, AAV, vaccinia virus or herpes virus.

109. The peptide of claim 106, wherein said peptide is attached to a solid support.

110. The peptide of claim 109, wherein the solid support is magnetic beads, Sepharose beads, agarose beads, a nitrocellulose membrane, a nylon membrane, a column chromatography matrix, a high performance liquid chromatography (HPLC) matrix or a fast performance liquid chromatography (PPLC) matrix.

111. A protein fusion construct comprising the peptide of claim 88 fused to a selected protein to form a protein fusion construct.

112. The protein fusion construct of claim 111, wherein the peptide is fused in frame at the amino or carboxy terminus of the selected protein.

113. The fusion construct of claim 24, wherein the selected protein is cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins.

114. The fusion construct of claim 113, wherein said selected protein is a pro-apoptosis agent selected from the group consisting of gramicidin, magainin, mellitin, defensin, cecropin, (KLAKLAK).sub.2 (SEQ ID NO:1), (KLAKKLA).sub.2 (SEQ ID NO:2), (KAAKKAA).sub.2 (SEQ ID NO:3) or (KLGKKLG).sub.3 (SEQ ID NO:4).

115. The fusion construct of claim 113, wherein said selected protein is an anti-angiogenic agent selected from the group consisting of thrombospondin, angiostatin, endostatin or pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-.beta., 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, paclitaxel, accutin, cidofovir, vincristine, bleomycin, AGM-1470, platelet factor 4 or minocycline.

116. The fusion construct of claim 113, wherein said selected protein is a cytokine selected from the group consisting of interleukin 1 (IL-1), IL-2, IL-5, IL-10, IL-11, IL-12, IL-18, interferon-γ (IF-γ), IF-α, IF-β, a tumor necrosis factor, and GM-CSF (granulocyte macrophage colony stimulating factor).

117. A method of preparing a VEGFR-1 targeted construct comprising obtaining a peptide in accordance with claim 1 and attaching the peptide to a molecule to prepare the construct.

118. A method of targeting the delivery of a molecule or protein to cells that express VEGFR-1, the method comprising the steps of:
(a) obtaining a peptide according to claim 98 or protein fusion construct according to claim 111; and
(b) administering the peptide or protein fusion construct to a cell population, wherein the population includes cells that express VEGFR-1, to thereby deliver the molecule or protein to said cells.

119. The method of claim 118, wherein the cells that express a VEGFR-1 are in a subject, the peptide or protein fusion construct is formulated in a pharmaceutically acceptable composition and the composition is administered to the subject.

120. The method of claim 119, wherein the subject is a human subject.

121. The method of claim 119, wherein the method is further defined as a detection method and the method further comprises detecting the peptide or protein that has been delivered to the cells.

122. The method of claim 119, wherein subject has a disease or disorder and the method is further defined as a therapeutic method.

123. The method of claim 122, subject has a disease or disorder with an angiogenesis component and the subject is in need of anti-angiogenesis therapy.

124. The method of claim 123, wherein the disease or disorder is cancer, diabetes, or arthritis.

125. The method of claim 124, wherein the disease is an angiogenic tumor.
